(19) United States
(12) Patent Application Publication
(10) Pub. No.: US 2019/0254577 A1
(43) Pub. Date: Aug. 22, 2019

(54) PHOTOACOUSTIC IMAGING APPARATUS, METHOD FOR ACQUIRING INFORMATION, AND NON-TRANSITORY COMPUTER-READABLE MEDIUM

(71) Applicant: CANON KABUSHIKI KAISHA, Tokyo (JP)

(72) Inventor: Yukio Furukawa, Sagamihara-shi (JP)

(21) Appl. No.: 16/347,709

(22) PCT Filed: Nov. 17, 2017
(86) PCT No.: PCT/JP2017/041461

§ 371 Date: May 6, 2019

(30) Foreign Application Priority Data
Nov. 24, 2016 (JP) 2016-228059

(51) Int. Cl. A61B 5/145; A61B 5/00; G01N 29/24

(52) U.S. Cl. CPC A61B 5/14542; G01N 29/2418; A61B 5/0095

(57) ABSTRACT

The present disclosure relates to a photoacoustic imaging apparatus. The photoacoustic imaging apparatus includes a light irradiation unit, a receiving unit, and a processing unit. The light irradiation unit irradiates an object with first intensity-modulated light of a first wavelength corresponding to a first coding sequence and second intensity-modulated light of a second wavelength corresponding to a second coding sequence different from the first coding sequence. The receiving unit outputs a first signal by receiving photoacoustic waves generated by irradiating the object with the first and second intensity-modulated light. The processing unit obtains at least one of a first decoded signal corresponding to the first wavelength and a second decoded signal corresponding to the second wavelength by performing a decoding process on the first signal based on information on the first and second coding sequences.

### TECHNICAL FIELD

The present disclosure relates to a photoacoustic imaging apparatus using a photoacoustic effect.

### BACKGROUND ART

Research on optical imaging apparatuses that apply light onto an object to image information on the interior of the object acquired on the basis of the irradiation light is being actively pursued in the medical field. One of the optical imaging techniques is photoacoustic tomography (PAT). In the PAT, light emitted from a light source is applied to an object, and acoustic waves generated from tissue that has absorbed the energy of the light propagated and diffused in the object are detected. The phenomenon of acoustic wave generation is referred to as a photoacoustic effect, and the generated acoustic waves are referred to as photoacoustic waves. The acoustic waves are generally ultrasonic waves.

PTL 1 discloses irradiation of an object with a pulse train in which a plurality of pulsed lights are arranged. PTL 1 also discloses reconstruction of image data based on photoacoustic waves generated in an object by light irradiation.

### CITATION LIST

### Patent Literature

### [PTL 1]

### Japanese Patent Laid-Open No. 2014-39801

### SUMMARY OF INVENTION

In the case of photoacoustic tomography using a plurality of times of light irradiation, typically, after reception of photoacoustic waves generated by light irradiation is completed, the next light irradiation is performed, and reception of photoacoustic waves that are generated next is started. However, with this method, the number of times of light irradiation per unit time is limited. This makes it difficult to improve the signal-to-noise (S/N) ratios of the received signals of the photoacoustic waves obtained per unit time.

### Solution to Problem

The present disclosure improves the S/N ratios of the received signals of photoacoustic waves obtained per unit time in a photoacoustic imaging apparatus using photoacoustic waves generated by a plurality of times of light irradiation.

The present disclosure provides a photoacoustic imaging apparatus. The photoacoustic imaging apparatus includes a light irradiation unit, a receiving unit, and a processing unit. The light irradiation unit irradiates an object with first intensity-modulated light of a first wavelength corresponding to a first coding sequence and second intensity-modulated light of a second wavelength corresponding to a second coding sequence different from the first coding sequence. The receiving unit outputs a first signal by receiving photoacoustic waves generated by irradiating the object with the first and second intensity-modulated light. The processing unit obtains at least one of a first decoded signal corresponding to the first wavelength and a second decoded signal corresponding to the second wavelength by performing a decoding process on the first signal based on information on the first and second coding sequences.

Further features of the present invention will become apparent from the following description of exemplary embodiments with reference to the attached drawings.

### BRIEF DESCRIPTION OF DRAWINGS

FIG. 1A is a graph schematically illustrating light intensity corresponding to a positive coding element and the received signal of the photoacoustic wave.

FIG. 1B is a graph schematically illustrating light intensity corresponding to a negative coding element and the received signal of the photoacoustic wave.

FIG. 2A is a graph schematically illustrating light intensity corresponding to a coding sequence and the received signal of the photoacoustic wave.

FIG. 2B is a graph schematically illustrating light intensity corresponding to a coding sequence and the received signal of the photoacoustic wave.

FIG. 2C is a graph schematically illustrating light intensity corresponding to a coding sequence and the received signal of the photoacoustic wave.

FIG. 3 is a block diagram of a photoacoustic imaging apparatus according to an embodiment of the present disclosure.

FIG. 4 is a block diagram illustrating the configuration of a computer and its peripherals according to an embodiment of the present disclosure.

FIG. 5A is a graph illustrating a characteristic of a semiconductor laser.

FIG. 5B is a graph illustrating a characteristic of the semiconductor laser.

FIG. 5C is a graph illustrating a characteristic of the semiconductor laser.

FIG. 6A is a graph illustrating a received signal corresponding to a positive coding element.

FIG. 6B is a graph illustrating the reception characteristic of a transducer.

FIG. 6C is a graph illustrating a signal received by the transducer in FIG. 6B.

FIG. 7 is a diagram illustrating the sequence of encoding according to a first embodiment.

FIG. 8A is a graph illustrating a driving signal according to the first embodiment.

FIG. 8B is a graph illustrating a received signal according to the first embodiment.

FIG. 8C is a graph illustrating a driving signal according to the first embodiment.

FIG. 8D is a graph illustrating a received signal according to the first embodiment.

FIG. 9 is a graph illustrating a received signal in which noise is added according to the first embodiment.

FIG. 10A is a graph illustrating a driving signal according to the first embodiment.

FIG. 10B is a graph illustrating a received signal according to the first embodiment.

FIG. 10C is a graph illustrating a driving signal according to the first embodiment.

FIG. 10D is a graph illustrating a received signal according to the first embodiment.

FIG. 11 is a graph illustrating another received signal in which noise is added according to the first embodiment.

FIG. 12A is a graph illustrating a decoded signal according to the first embodiment.

FIG. 12B is a graph illustrating a decoded signal according to the first embodiment.

FIG. 12C is a graph illustrating a decoded signal according to the first embodiment.

FIG. 13A is a graph illustrating another decoded signal according to the first embodiment.

FIG. 13B is a graph illustrating another decoded signal according to the first embodiment.

FIG. 13C is a graph illustrating another decoded signal according to the first embodiment.

FIG. 14 is a diagram illustrating the configuration of a driving unit according to the first embodiment.

FIG. 15 is a block diagram of a photoacoustic imaging apparatus according to a second embodiment.

FIG. 16 is a diagram illustrating the sequence of encoding according to a second embodiment.

FIG. 17A is a graph illustrating a received signal according to the second embodiment.

FIG. 17B is a graph illustrating a received signal according to the second embodiment.

FIG. 17C is a graph illustrating a received signal according to the second embodiment.

FIG. 17D is a graph illustrating a received signal according to the second embodiment.

FIG. 18A is a graph illustrating another received signal according to the second embodiment.

FIG. 18B is a graph illustrating another received signal according to the second embodiment.

FIG. 18C is a graph illustrating another received signal according to the second embodiment.

FIG. 18D is a graph illustrating another received signal according to the second embodiment.

FIG. 19A is a graph illustrating another received signal according to the second embodiment.

FIG. 19B is a graph illustrating another received signal according to the second embodiment.

FIG. 19C is a graph illustrating another received signal according to the second embodiment.

FIG. 19D is a graph illustrating another received signal according to the second embodiment.

FIG. 20A is a graph illustrating another received signal according to the second embodiment.

FIG. 20B is a graph illustrating another received signal according to the second embodiment.

FIG. 20C is a graph illustrating another received signal according to the second embodiment.

FIG. 20D is a graph illustrating another received signal according to the second embodiment.

FIG. 21A is a graph illustrating a decoded signal according to the second embodiment.

FIG. 21B is a graph illustrating a decoded signal according to the second embodiment.

FIG. 21C is a graph illustrating a decoded signal according to the second embodiment.

FIG. 21D is a graph illustrating a decoded signal according to the second embodiment.

FIG. 21E is a graph illustrating a decoded signal according to the second embodiment.

FIG. 22A is a graph illustrating another decoded signal according to the second embodiment.

FIG. 22B is a graph illustrating another decoded signal according to the second embodiment.

FIG. 22C is a graph illustrating another decoded signal according to the second embodiment.

FIG. 22D is a graph illustrating another decoded signal according to the second embodiment.

FIG. 22E is a graph illustrating another decoded signal according to the second embodiment.

FIG. 23A is a graph illustrating another decoded signal according to the second embodiment.

FIG. 23B is a graph illustrating another decoded signal according to the second embodiment.

FIG. 23C is a graph illustrating another decoded signal according to the second embodiment.

FIG. 23D is a graph illustrating another decoded signal according to the second embodiment.

FIG. 23E is a graph illustrating another decoded signal according to the second embodiment.

### DESCRIPTION OF EMBODIMENTS

It is known in the art that, when light is applied to a substance, acoustic waves (also referred to as photoacoustic waves) are generated owing to a photoacoustic effect. Furthermore, it is known that the concentration of the components of a substance can be calculated as spectral information on the basis of photoacoustic waves generated by irradiating the substance with lights of a plurality of different wavelengths. In particular, it is known that the hemoglobin concentration in blood can be calculated on the basis of photoacoustic waves generated by irradiating the blood with lights of a different plurality of wavelengths using the fact that the optical absorption coefficient spectrum of blood depends on hemoglobin concentration.

However, in the case of using lights of a plurality of wavelengths, if the period of irradiation with light of a certain wavelength and the period of irradiation with light of another wavelength overlap, it cannot be determined from which of photoacoustic waves the received signal is obtained. To address it, there is a known method of separating received signals in terms of time such that after photoacoustic waves due to light of a certain wavelength are received, photoacoustic waves due to light of another wavelength are received. However, with the method of separating received signals corresponding to the lights of different wavelengths by ensuring a sufficient time between the irradiation of light of different wavelengths, the signal-to-noise (S/N) ratio of the received signals of photoacoustic waves obtained per unit time decreases.

The present disclosure has found out a method of encoding by irradiating a substance with light of a certain wavelength in the form of intensity-modulated light corresponding to a certain coding sequence and irradiating the substance with light of another wavelength in the form of intensity-modulated light corresponding to another coding sequence. By decoding the thus-encoded received signals of the photoacoustic waves using information on the coding sequences used in encoding, decoded signals corresponding to the individual wavelengths can be obtained. Such encoding and decoding allows the received signals corresponding to the individual wavelengths to be separated even if the irradiation period of the wavelengths overlap. This improves the S/N ratio of the received signals of photoacoustic waves obtained per unit time.

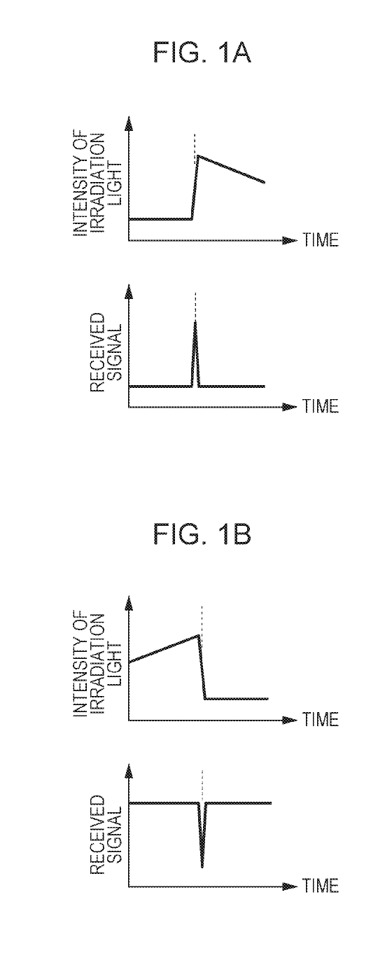

A method of encoding based on a coding sequence including positive and negative coding elements by controlling irradiation light using a photoacoustic imaging apparatus that processes the received signals of the photoacoustic waves will be described. FIGS. 1A and 1B are graphs that schematically show temporal changes in the intensity of irradiation light and the level of the received signal of a photoacoustic wave generated by the irradiation light. Typically, as illustrated in FIG. 1A, when the temporal change in the intensity of the irradiation light is positive, a positive-level received signal can be obtained. In contrast, as illustrated in FIG. 1B, when the temporal change in the intensity of the irradiation light is negative, a negative-level received signal can be obtained. The level of the received signal increases as the change in the intensity of the irradiation light per unit time increases. In FIGS. 1A and 1B, the propagation time of photoacoustic waves from a sound source to a receiving unit is ignored.

The inventor has found out the present disclosure from the conception that the positive and negative of the level of the received signal can be controlled by controlling the positive and negative of a temporal change in the intensity of the irradiation light, as illustrated in FIGS. 1A and 1B. In other words, the inventor has found out that the positive and negative of coding elements constituting a coding sequence in encoding by controlling the positive and negative of a temporal change in the intensity of the irradiation light. For example, a coding sequence including positive and negative coding elements can be defined by combining light irradiation illustrated in FIG. 1A at which the coding element is {1} and light irradiation illustrated in FIG. 1B at which the coding elements is {-1}. In this specification, light for generating a photoacoustic wave corresponding to a positive coding element is referred to as “positive intensity-modulated light”. Light for generating a photoacoustic wave corresponding to a negative coding element is referred to as “negative intensity-modulated light”.

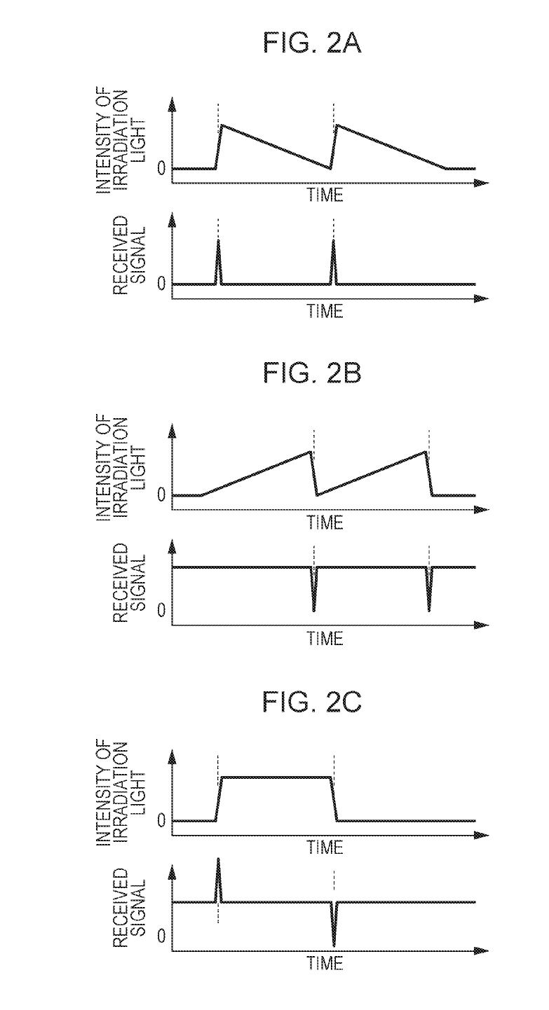

Examples of sequences of light irradiation corresponding to the coding sequences of several patterns will be described with reference to FIGS. 2A to 2C. The dotted lines in FIGS. 2A to 2C each indicate the reference timing of each coding element.

FIG. 2A is a graph that schematically illustrates temporal changes in the intensity of irradiation light and the level of the received signal of the photoacoustic wave corresponding to a coding sequence {1, 1}. The sequence of the irradiation light illustrated in FIG. 2A includes two consecutive lights (positive intensity-modulated lights) whose intensity sharply increases in a short time and then gradually decreases with time. The timing at which the intensity increases sharply in a short time is adjusted to a reference timing corresponding to a positive coding element. For example, the timing at the center of the period during which the intensity sharply increases in a short time can be made to coincide with the reference timing. In this case a large positive received signal can be obtained at the reference timing. The large positive received signal is a signal corresponding to the positive coding element {1}.

FIG. 2B is a graph that schematically illustrates temporal changes in the intensity of irradiation light and the level of the received signal of the photoacoustic wave corresponding to a coding sequence {−1, −1}. The sequence of the irradiation light illustrated in FIG. 2B includes two consecutive lights (negative intensity-modulated lights) whose intensity gradually increases with time and sharply decreases in a short time. The timing at which the intensity sharply decreases in a short time is adjusted to a reference timing corresponding to a negative coding element. Specifically, the timing at the center of the period during which the intensity sharply decreases in a short time is made to coincide with the reference timing. In this case a large negative received signal can be obtained at the reference timing. The large negative received signal is a signal corresponding to the negative coding element {−1}.

FIG. 2C is a graph that schematically illustrates temporal changes in the intensity of irradiation light and the level of the received signal of the photoacoustic wave corresponding to a coding sequence {1, −1}. The sequence of the irradiation light illustrated in FIG. 2C is such that the positive intensity-modulated light illustrated in FIG. 2A is applied, and then the negative intensity-modulated light illustrated in FIG. 2B is applied. The irradiation timing is controlled so that the timing at which the intensity of the positive intensity-modulated light sharply increases corresponds to the reference timing of the positive coding element {1}, and the timing at which the intensity of the negative intensity-modulated light sharply decreases corresponds to the reference timing of the negative coding element {−1}. In this case, a large positive received signal and a large negative received signal can be obtained at the respective reference timings.

Since the portion of the positive intensity-modulated light that gradually decreases with time and the portion of the negative intensity-modulated light that gradually increases with time overlap in time, the overlapping portion forms a rectangular wave as a result. Thus, when the positive and negative coding elements of the coding sequence are next to each other, an unnecessary photoacoustic wave is not generated by setting the light intensity between the reference timings substantially constant. This allows high-accuracy encoding using light irradiation. Although FIG. 2C illustrates an example of the coding sequence {1, −1}, the light intensity between the reference timings may be set substantially constant also for a coding sequence {−1, 1}. The temporal change in the light intensity between the reference timings may be assumed to be substantially constant as long as the temporal change is within a predetermined range in which a photoacoustic wave having a frequency out of the reception band of a transducer that receives the photoacoustic wave is generated. In FIGS. 2A to 2C, the propagation time of photoacoustic waves from a sound source to a receiving unit is ignored.

Thus, decoding accuracy of decoding based on a coding sequence including positive and negative coding elements can be increased by performing light irradiation corresponding the coding sequence including positive and negative coding elements and encoding for the positive and negative coding elements. In particular, for semiconductor lasers and light-emitting diodes (LEDs) whose output light intensities are smaller than those of high-output light sources, such as a solid-state laser, the S/N ratio of the received signal is desirably improved by increasing the number of irradiation times per unit time. In such a case, by executing light irradiation for encoding on the basis of a coding sequence including positive and negative coding elements before reception of a photoacoustic wave generated first is completed, decoded signals with a high S/N ratio can be accurately obtained.

The acoustic waves generated owing to a photoacoustic effect according to an embodiment of the present disclosure is typically ultrasonic waves including sound waves and acoustic waves. The present disclosure can be applied to a photoacoustic imaging apparatus that obtains image data on the basis of photoacoustic waves generated by the photoacoustic effect.

A photographic image acquired by the photoacoustic imaging apparatus according to an embodiment of the present disclosure includes all images derived from photoacoustic waves generated by light irradiation. The photographic image is image data indicating the spatial distribution of at least one of object information such as the generated sound pressure (initial sound pressure), the optical absorption energy density, and the optical absorption coefficient of the photoacoustic waves, and the concentration (the degree of oxygen saturation) of a substance constituting the object. Object information acquired on the basis of photoacoustic waves generated by irradiation of light of a different plurality of wavelengths is spectral information including the concentration of substances constituting the object.

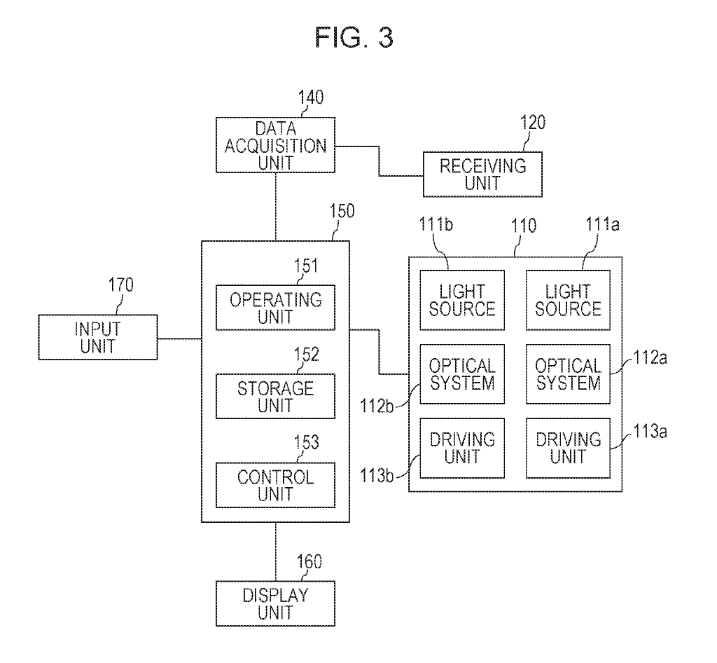

Referring to FIG. 3, the configuration of the photoacoustic imaging apparatus according to the present embodiment will be described hereinbelow. FIG. 3 is a schematic block diagram of the overall photoacoustic imaging apparatus. The photoacoustic imaging apparatus according to the present embodiment includes a light irradiation unit 110, a receiving unit 120, a data acquisition unit 140, a computer 150, a display unit 160, and an input unit 170.

The light irradiation unit 110 irradiates an object 100 with light, so that acoustic waves are generated from the object 100. The acoustic waves generated by a photoacoustic effect due to light are also referred to as photoacoustic waves. The receiving unit 120 receives the photoacoustic waves and outputs an electrical signal (a photoacoustic signal) which is an analog signal.

The data acquisition unit 140 converts the analog signal output from the receiving unit 120 to a digital signal and outputs the digital signal to the computer 150. The computer 150 stores the digital signal output from the data acquisition unit 140 as signal data derived from the photoacoustic wave.

The computer 150 serving as a processing unit performs processing (described later) on the stored digital signal to generate image data indicating a photographic image. The computer 150 also performs image processing on the acquired image data for display and thereafter outputs the image data to the display unit 160. The display unit 160 displays the photographic image. The user, such as a doctor or an operator, can make a diagnosis by checking the photographic image displayed on the display unit 160. The display image is stored in a memory in the computer 150 or a data management system connected to the modality over a network on the basis of a storage instruction from the user or the computer 150.

The computer 150 also controls driving of the components of the photoacoustic imaging apparatus. The display unit 160 may also display a graphic user interface (GUI) in addition to images generated by the computer 150. The input unit 170 is configured so that the user can input information. The user can perform operations such as instructions to start and terminate measurement and store a generated image.

The details of the components of the photoacoustic imaging apparatus according to the present embodiment will be described hereinbelow.

### [Light Irradiation Unit 110]

The light irradiation unit 110 includes a first light source 111a and a second light source 111b as two light sources of different wavelengths. The light irradiation unit 110 further includes a first optical system 112a and a second optical system 112b that respectively guide the light emitted from the first light source 111a and the second light source 111b to the object 100. The light irradiation unit 110 further includes a first driving unit 113a and a second driving unit 113b that respectively control driving of the first light source 111a and the second light source 111b.

The light emitted from the first and second light sources 111a and 111b may have a pulse width of 1 ns or more and 100 ns or less. The light may have a wavelength within a range of about 400 nm to 1,600 nm. For imaging blood vessels at high resolution, light having a wavelength (400 nm or more and 700 nm or less) with large absorptance in blood vessels may be used. For imaging a deep part of a living organism, light having a wavelength (700 nm or more and 1,100 nm or less) with typically small absorptance in background tissue (water, fat, etc.) of a living organism may be used.

Examples of the first and second light sources 111a and 111b include a laser and a light-emitting diode (LED). A light source capable of changing the wavelength of light may also be used.

For example, the first and second light sources 111a and 111b may be semiconductor lasers or LEDs capable of emitting light following a saw tooth driving waveform (a driving current) with a frequency of 1 MHz or more.

Examples of the first and second optical systems 112a and 112b include optical elements, such as a lens, a mirror, and an optical fiber. If the object 100 is a breast or the like, the light output units of the first and second optical systems 112a and 112b may be diffuser panels for diffusing light to increase the beam diameter of the pulsed light. For a photoacoustic microscope, the light output units of the first and second optical systems 112a and 112b may be lenses and focus the beam to increase the resolution. The light irradiation unit 110 may apply light directly from the first and second light sources 111a and 111b to the object 100 without the first and second optical systems 112a and 112b.

The first and second driving units 113a and 113b generate driving current (current to be input to the first and second light sources 111a and 111b) for driving the first and second light sources 111a and 111b. The first and second driving units 113a and 113b may use a power source capable of temporally varying the current to be input to the first and second light sources 111a and 111b. By controlling the output of the first and second light sources 111a and 111b with the first and second driving units 113a and 113b, the light as illustrated in FIGS. 1A and 1B is generated to achieve encoding. The first and second driving units 113a and 113b may be controlled by a control unit 153 (described later) in the computer 150. The first and second driving units 113a and 113b may include a control unit for controlling the current value so that the control unit can control the input current. The relationship between the driving current and the intensity of the irradiation light will be described later. [Receiving Unit 120]

The receiving unit 120 includes a transducer that outputs an electrical signal by receiving acoustic waves and a supporter that supports the transducer.

Examples of a constituent member of the transducer include a piezoelectric ceramic material typified by piezoelectric zirconate titanate (PZT) (lead zirconate titanate) and a high-molecule piezoelectric membrane material typified by polyvinylidene fluoride (PVDF). Other elements other than the piezoelectric element may be used. For example, a capacitive micro-machined ultrasonic transducer (CMUT) or a transducer with a Fabry-Perot interferometer can be used. Any transducers capable of outputting an electrical signal by receiving acoustic waves can be employed. The signal obtained by the transducer is a time-resolved signal. In other words, the amplitude of the signal obtained by the transducer indicates a value based on a sound pressure received by the transducer at each time (for example a value proportional to the sound pressure).

A frequency component constituting the photoacoustic wave typically ranges from 100 KHz to 100 MHz, and a transducer capable of detecting these frequencies can be employed.

The supporter may be a supporter that supports a plurality of transducers on a flat surface or a curved surface called a 1D array, a 1.5D array, a 1.75D array, or a 2D array. An array in which a plurality of transducers are arranged in a curved surface is also referred to as a three-dimensional transducer array.

The receiving unit 120 may further include an amplifier that amplifies time-series analog signals output from the transducer. The receiving unit 120 may further include an analog-to-digital converter that converts time-series analog signals output from the transducer to time-series digital signals. In other words, the receiving unit 120 may include the data acquisition unit 140 (described later).

A plurality of transducers may be ideally disposed around the object 100 to detect acoustic waves at various angles. However, if the object 100 is so large that the transducers cannot be disposed so as to surround the entire periphery thereof, the transducers may be disposed on a hemispherical supporter to substantially surround the entire periphery. The arrangement and number of the transducers and the shape of the supporter may be optimized according to the object, and any type of receiving unit 120 can be employed in the present disclosure.

The space between the receiving unit 120 and the object 100 may be filled with a medium capable of propagating photoacoustic waves. An example of this medium is a material in which acoustic waves can propagate and the acoustic characteristics match at the interface with the object 100 and the transducers and which has the highest possible transmittance of the photoacoustic waves. For example, this medium can be water or ultrasonic wave gel.

In the case where the apparatus according to the present embodiment generates an ultrasonic image, in addition to the photographic image, by transmitting and receiving acoustic waves, the transducer may also function as a transmitting unit that transmits the acoustic waves. The transducer serving as a receiving unit and the transducer serving as a transmitting unit may be a single (common) transducer or separate transducers.

### [Data Acquisition Unit 140]

The data acquisition unit 140 includes an amplifier that amplifies an electrical signal, which is an analog signal output from the receiving unit 120, and an analog-to-digital converter that converts the analog signal output from the amplifier to a digital signal. The data acquisition unit 140 may be formed of a field programmable gate array (FPGA) chip. The digital signal output from the data acquisition unit 140 is stored in a storage unit 152 in the computer 150. The data acquisition unit 140 is also referred to as a data acquisition system (DAS). In this specification, the electrical signal includes an analog signal and a digital signal. The data acquisition unit 140 is connected to a light sensor mounted to the light output unit of the light irradiation unit 110. The data acquisition unit 140 may start processing in synchronism with emission of light from the light irradiation unit 110. Alternatively, the data acquisition unit 140 may start the processing in synchronism with an instruction given using a freeze button or the like.

### [Computer 150]

The computer 150 serving as an information processing unit includes an operating unit 151, the storage unit 152, and the control unit 153. The function of each component will be described in the description of the process flow.

A unit responsible for an operating function as the operating unit 151 may include a processor, such as a central processing unit (CPU) or a graphics processing unit (GPU), or an operating circuit, such as a field programmable gate array (FPGA) chip. The unit may include either a single processor or operating circuit or a plurality of processor or operating circuits. The operating unit 151 may process the received signal using various parameters, such as the sound velocity of the object and the sound velocity of a medium in which the acoustic wave propagates, sent from the input unit 170.

The storage unit 152 may be a non-transitory storage medium, such as a read only memory (ROM), a magnetic disk, or a flash memory. The storage unit 152 may be a volatile medium, such as a random access memory (RAM). A storage medium in which programs are stored is a non-transitory storage medium. The storage unit 152 may include either one storage medium or a plurality of storage media.

The storage unit 152 can store image data indicating a photographic image generated by the operating unit 151 using a method described later.

The control unit 153 includes an operating element, such as a CPU. The control unit 153 controls the operation of each component of the photoacoustic imaging apparatus. The control unit 153 may control each component of the photoacoustic imaging apparatus by receiving instruction signals to start measurement or the like according to various operations from the input unit 170. The control unit 153 reads program codes stored in the storage unit 152 and controls the operations of the components of the photoacoustic imaging apparatus.

The computer 150 may be a dedicated workstation. The components of the computer 150 may be different pieces of hardware. At least part of the components of the computer 150 may be a single piece of hardware.

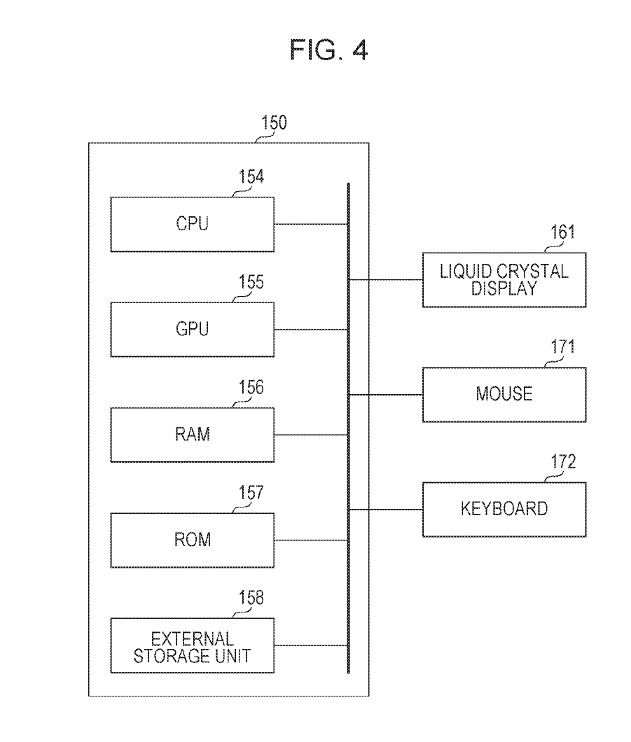

FIG. 4 illustrates a specific configuration example of the computer 150 according to the present embodiment. The computer 150 according to the present embodiment includes a CPU 154, a GPU 155, a RAM 156, a ROM 157, and an external storage unit 158. The computer 150 connects to a liquid crystal display 161 serving as the display unit 160 and a mouse 171 and a keyboard 172 serving as the input unit 170.

The computer 150 and the receiving unit 120 may be housed in a common casing. Part of signal processing may be performed by a computer housed in the casing, and the remaining signal processing may be performed by a computer disposed outside the casing. In this case, the computers disposed inside and outside the casing can be collectively referred to as a computer according to the present embodiment. In other words, the hardware constituting the computer may not be housed in one casing.

### [Display Unit 160]

Examples of the display unit 160 include a liquid crystal display and an organic electro luminescence (EL) display. The display unit 160 displays images and the value of a specified position based on object information obtained by the computer 150. The display unit 160 may display a GUI for operating images and the apparatus. The object information may be displayed after image processing (adjustment of the luminance value, etc.) is performed on the display unit 160 or the computer 150.

### [Input Unit 170]

The input unit 170 may be an operator operable console, such as a mouse and a keyboard. The display unit 160 may be a touch panel serving as the input unit 170.

The components of the photoacoustic imaging apparatus may be either separate units or a single unit. A least part of the components of the photoacoustic imaging apparatus may be a single unit.

### [Object 100]

The object 100, which is not a component of the photoacoustic imaging apparatus, will be described below. The photoacoustic imaging apparatus according to the present embodiment can be used to diagnose malignant tumors, vascular diseases, and so on of humans and animals and to follow up on chemotherapy. Therefore, conceivable examples of the object 100 include diagnostic target parts, such as the breasts, the organs, the vascular networks, the head, the neck, the abdomen, the extremities including fingers and toes of human bodies and animals. For example, for a human body, oxyhemoglobin, deoxyhemoglobin, blood vessels containing much oxyhemoglobin or deoxyhemoglobin or new blood vessels formed in the vicinity of tumor may be the target light absorber. Furthermore, plaque on a carotid artery wall may be the target light absorber. Pigments, such as methylene blue (MB) and indocyanine green (ICG), gold fine particles, or externally introduced substances in which the above pigments or gold fine particles are accumulated or chemically modified may be the light absorber. A puncture needle or a light absorber put on a puncture needle may be the observation target.

### [Received Signal of Photoacoustic Wave Corresponding to Coding Element {1}]

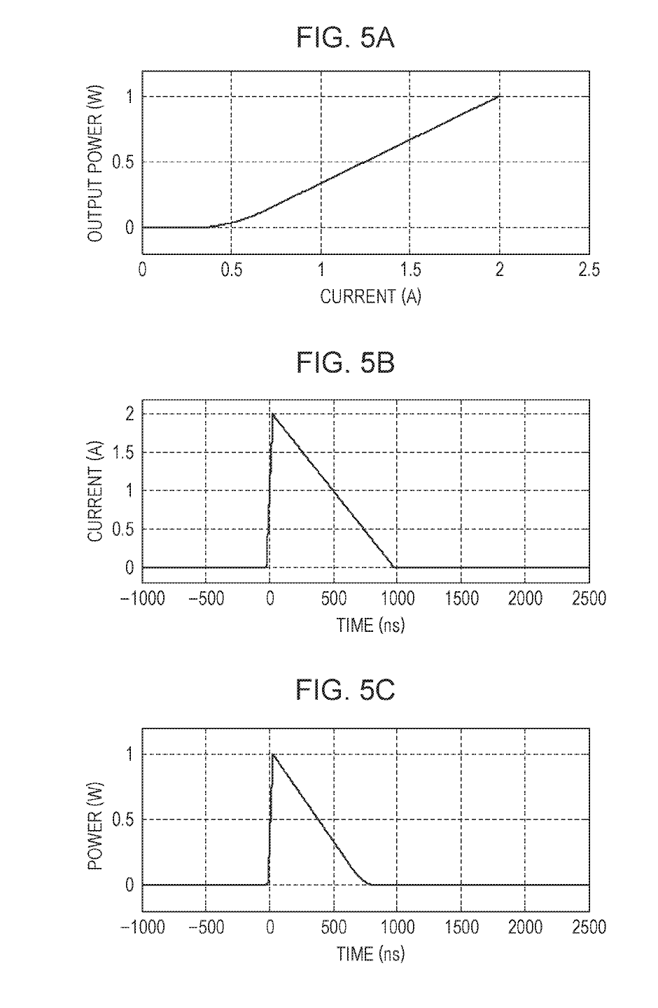

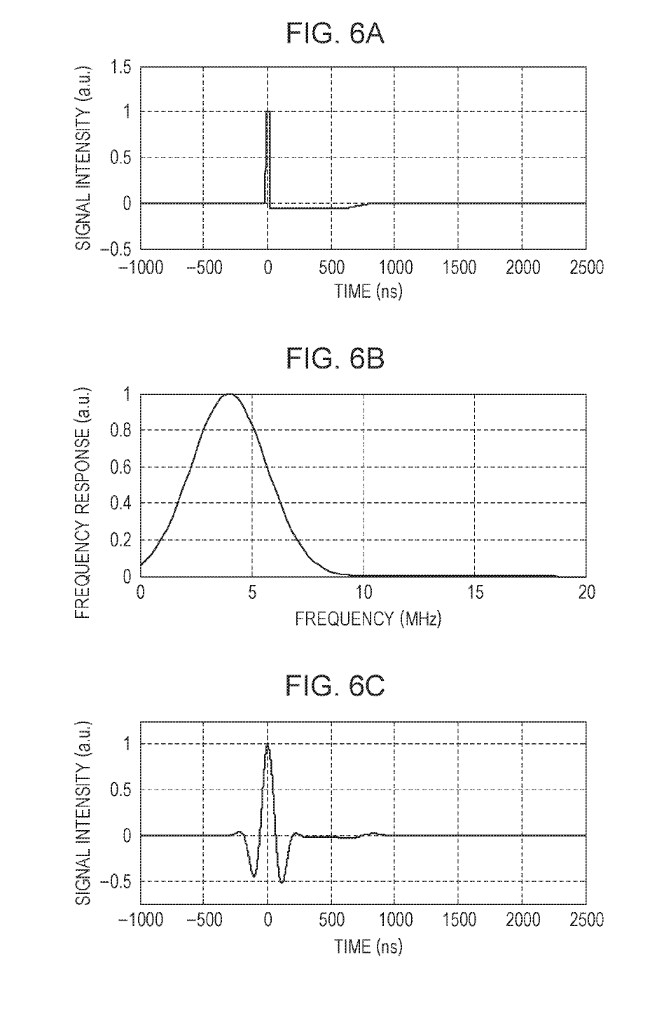

Irradiation light and the received signal of a photoacoustic wave corresponding to each coding element when the photoacoustic imaging apparatus according to the present embodiment is used will be considered. Referring to FIGS. 5A to 5C and FIGS. 6A to 6C, irradiation light and the received signal of a photoacoustic wave corresponding to a coding element {1} will be described. The data illustrated in FIGS. 5A to 6C and FIGS. 6A to 6C is data obtained by simulation.

FIG. 5A is a diagram illustrating the current-light output characteristic of a semiconductor laser serving as the first light source 111a or the second light source 111b. The threshold current of the semiconductor laser is 0.5 A, and the light output when a current of 2 A is input is 1 W. In the case of the semiconductor laser, the current-light output characteristic is typically substantially linear in a region where the current is equal to or greater than the threshold current. In other words, with the semiconductor laser, the temporal waveform of the input current is the temporal waveform of the light output (the intensity of the irradiation light).

FIG. 5B illustrates a driving current (a first driving current) for generating light corresponding to a positive coding element, in which the current value is increased from 0 A to 2 A in the time of 50 ns and is decreased from 2 A to 0 A in the time of 950 ns. In other words, as compared with the temporal change of the current at the timing corresponding to the positive coding element of the first driving current, the temporal change of the current at the other timing is smaller. Thus, the temporal change of the positive intensity-modulated light corresponding to the positive coding element at the reference timing corresponding to the positive coding element is larger than the temporal change of the light intensity at the other timing.

FIG. 5C illustrates light output when the semiconductor laser is driven by the driving current in FIG. 5B. This shows that the light is output substantially linearly with respect to the driving current, as described above.

FIG. 6A illustrates a received signal in the case where photoacoustic waves generated when a point-like light absorber is irradiated with light are received by a transducer with an infinite reception band. This is equal to the time derivative of the light output curve in FIG. 5C. Thus, a large positive received signal is obtained in accordance with a sharp rise in light output in a short time.

However, the reception band of the transducer is actually unlikely to be infinite and has some frequency characteristics. FIG. 6B illustrates the reception characteristics of a transducer having frequency characteristics of a central frequency of 4 MHz and frequencies of 2 to 6 MHz in a 6-dB band. FIG. 6C illustrates a received signal when photoacoustic waves generated by the light irradiation in FIG. 5C are received by a transducer having the reception characteristics illustrated in FIG. 6B. Thus, a large positive received signal can be obtained in correspondence with a sharp rise in light output in a short time even in consideration of the reception characteristic of the transducer. This large positive received signal is a received signal corresponding to a positive coding element (for example, the coding element {1}).

In FIGS. 6A to 6C, the time of propagation of the photoacoustic waves from the sound source to the receiving unit is ignored.

### [Photoacoustic Wave Corresponding to Code Element {−1}]

The received signal of a photoacoustic wave obtained by driving the semiconductor laser with a driving current (a second driving current) which is a current inverted from the driving current in FIG. 5B on the time axis has a waveform in which the received signal in FIG. 6C is inverted on the time axis, and the plus-minus sign of the signal level is inverted (a detailed description thereof will be omitted). In other words, as compared with the temporal change of the current at the timing corresponding to the negative coding element of the second driving current, the temporal change of the current at the other timing is smaller. Thus, the temporal change of the negative intensity-modulated light corresponding to the negative coding element at the reference timing corresponding to the negative coding element is larger than the temporal change of the light intensity at the other timing. The thus-obtained large negative received signal is a received signal corresponding to a negative coding element (for example, a coding element {−1}).

### [Sequence of Generation of Photographic Image]

Next, a method for generating a photographic image by encoding and decoding (a method for acquiring information) using the photoacoustic imaging apparatus according to the present embodiment will be described.
S1. The light irradiation unit 110 applies light of a first wavelength encoded with the first coding sequence and light of a second wavelength encoded with the second coding sequence.
S2. A plurality of transducers in the receiving unit 120 receive photoacoustic waves generated owing to the coded light and output first received signals.
S3. The light irradiation unit 110 applies light of the first wavelength encoded with a third coding sequence and the light of the second wavelength encoded with a fourth coding sequence.
S4. The plurality of transducers in the receiving unit 120 receive photoacoustic waves generated from the coded light and output first received signals.
S5. The operating unit 151 performs a decoding process on the first and second received signals output from the plurality of transducers to generate a received signal (decoded signal) decoded for each transducer.
S6. The operating unit 151 generates a photoacoustic image using the plurality of decoded signals corresponding to the plurality of transducers.

A specific method of encoding and decoding will be described later in the embodiments.

### [Reconstruction Technique]

The operating unit 151 can generate image data by performing back projection (simple back projection) of a plurality of decoded signals on a calculation space. In other words, the operating unit 151 may convert decoded signals, which are time signals, to spatial distribution data. For example, the operating unit 151 may obtain image data (one line of image data) that is linear in a depthwise direction by performing phasing addition on a plurality of decoded signals. The operating unit 151 may generate two-dimensional or three-dimensional image data by executing this process on a plurality of lines. The operating unit 151 may perform envelope processing on the spatial distribution data obtained by the phasing addition to generate image data.

A known PAT image reconstruction method is a universal back projection (UBP) method. This is a method for acquiring a photographic image by back-projecting data obtained by temporally differentiating received signals obtained by the receiving unit and inverting the plus-minus sign. This method can be employed in the case where a photoacoustic wave generated when impulse-like pulsed light is applied has an alphabetical N shape called N-shape.

It can be understood that the photoacoustic wave generated in the present embodiment is a photoacoustic wave in which the former half and the latter half of the N-shape are separated, and the former half is a photoacoustic wave corresponding to the coding element {1}, and the latter half is a photoacoustic wave corresponding to the coding element {−1}. For that reason, the coded and decoded received signals as in the present embodiment cannot yield a correct result even using the UBP method. Therefore, in the present embodiment, back projection may be performed by performing a phasing addition process of phasing and then adding the decoded received signals without performing the preprocessing on the decoded received signals, which is performed in the UBP method. In this specification, the reconstruction method of performing back projection without executing the preprocessing on the decoded received signals in the UBP method is referred to as simple back projection. Other reconstruction algorithms for converting signal data to three-dimensional volume data include back projection in the time domain, back projection in the Fourier domain, a model-based method (a repeated calculation method), and any other methods.

### First Embodiment

### [Encoding and Decoding Using Complementary Code]

Encoding and decoding by light irradiation using complementary codes will be described.

A set of two coding sequences {aᵢ} and {cᵢ}, (i=1 to N, where N is a code length, each coding element is 1 or −1) with a code length of N in which the sum of their autocorrelation functions are 2N at the peak, and is 0 at the other all points other than the peak are referred to as complementary codes.

The autocorrelation function is expressed as:

$$\begin{matrix}[ Math . 1 ] & \\ {( a * a )}_{m} = {\sum }_{i = 1}^{N} {a}_{i + m} \cdot {a}_{i} where m = ( - N + 1 ) to ( N - 1 ) & ( 1 )\end{matrix}$$

A set of autocorrelation functions {aᵢ}={1, 1} and {cᵢ}={1, −1} are complementary codes.

That is, (a*a)={1, 2, 1}, (c*c)={−1, 2, −1}, (a*a)+(c*c)={0, 4, 0}

It is known that complementary codes are present in a code sequence of a code length of n-th power of 2 or n-th power of 5×2 (n is a natural number).

A set of coding sequences that are complementary codes include complementary codes whose sum of cross-correlation functions is 0. The relationship in which the sum of the cross-correlation functions in two sets of complementary codes is referred to as “complete orthogonal relationship” for convenience sake.

The cross-correlation function is expressed as:

$$\begin{matrix}[ Math . 2 ] & \\ {( a * c )}_{m} = {\sum }_{i = 1}^{N} {a}_{i + m} \cdot {c}_{i} where m = ( - N + 1 ) to ( N - 1 ) & ( 2 )\end{matrix}$$

For example, a set of a second coding sequence {bᵢ}={1, −1} and a fourth coding sequence {dᵢ}={1, 1} satisfies “complete orthogonal relationship” with respect to a set of a first coding sequence {aᵢ}={1, 1} and a third coding sequence {cᵢ}={1, −1} which are complementary codes. That is, (a*b)={−1, 0, 1}, (c*d)={1, 0, −1}, and (a*b)+(c*d)=0 hold. At that time, (b*a)+(d*c)=0 also holds.

In the case of a code length of 8, if a first coding sequence {aᵢ}={1, 1, −1, 1, −1, −1, −1, 1}, a second coding sequence {bᵢ}={1, 1, −1, 1, 1, 1, 1, −1}, a third coding sequence {cᵢ}={1, −1, −1, −1, −1, 1, −1, −1}, and a fourth coding sequence {dᵢ}={1, −1, −1, −1, 1, −1, 1, 1}, then (a*a)={1, 0, −3, 0, −1, 0, −1, 8, −1, 0, −1, 0, −3, 0, 1}, (c*c)={−1, 0, 3, 0, 1, 0, 1, 8, 1, 0, 1, 0, 3, 0, −1}, and (a*a)+(c*c)={0, 0, 0, 0, 0, 0, 0, 16, 0, 0, 0, 0, 0, 0, 0} hold, so that a set of the first coding sequence {aᵢ} and the third coding sequence {cᵢ} are complementary codes. Likewise,

(b*b)={−1, 0, 3, 0, 1, 0, 1, 8, 1, 0, 1, 0, 3, 0, −1},
(d*d)={1, 0, −3, 0, −1, 0, −1, 8, −1, 0, −1, 0, −3, 0, 1}, and
(b*b)+(d*d)={0, 0, 0, 0, 0, 0, 0, 16, 0, 0, 0, 0, 0, 0, 0} hold, so that a set of the second coding sequence {bᵢ} and the fourth coding sequence {dᵢ} are also complementary codes. Furthermore,
(a*b)={−1, 0, 3, 0, 3, 0, −1, 0, −3, 0, 1, 0, −3, 0, 1},
(c*d)={1, 0, −3, 0, −3, 0, 1, 0, 3, 0, −1, 0, 3, 0, −1},
(a*b)+(c*d)=0,
(b*a)={1, 0, −3, 0, 1, 0, −3, 0, −1, 0, 3, 0, 3, 0, −1},
(d*c)={−1, 0, 3, 0, −1, 0, 3, 0, 1, 0, −3, 0, −3, 0, 1}, and
(b*a)+(d*c)=0 hold, so that a set of the coding sequences {aᵢ} and {cᵢ}, which are first complementary codes, and a set of the coding sequences {bᵢ} and {dᵢ}, which are second complementary codes, satisfy “complete orthogonal relationship”.

Thus, using a set of coding sequences that satisfies “complete orthogonal relationship” enables the following.
When a signal A encoded with the first coding sequence {aᵢ} and a signal C encoded with the third coding sequence {cᵢ} are present, the sum of a signal obtained by decoding the signal A with the first coding sequence {aᵢ} and a signal obtained by decoding the signal C with the third coding sequence {cᵢ} is a delta function.
The sum of a signal obtained by decoding the signal A with the second coding sequence {bᵢ} and a signal obtained by decoding the signal C with the fourth coding sequence {dᵢ} is zero.
When a signal B encoded with the second coding sequence {bᵢ} and a signal D encoded with the fourth coding sequence {dᵢ} are present, the sum of a signal obtained by decoding the signal B with the second coding sequence {bᵢ} and a signal obtained by decoding the signal D with the fourth coding sequence {dᵢ} is a delta function.
The sum of a signal obtained by decoding the signal B with the first coding sequence {aᵢ} and a signal obtained by decoding the signal D with the third coding sequence {cᵢ} is zero.

Such coding sequences are used in a photoacoustic imaging apparatus that uses lights of two wavelengths, and light of the first wavelength is encoded with the first coding sequence {aᵢ} and the third coding sequence {cᵢ}, and light of the second wavelength is encoded with the second coding sequence {bᵢ} and the fourth coding sequence {dᵢ}. In this case, even if the light irradiation times overlap, signals obtained from the light of the first wavelength and signals obtained from the light of the second wavelength can be separately acquired by a decoding process.

A case in which lights of two different wavelengths are used as irradiation light by the photoacoustic imaging apparatus illustrated in FIG. 3 will be described. In the present embodiment, the first light source 111a is a semiconductor laser having a wavelength 755 nm (a first wavelength Δ1) and a maximum light output of 1 W. The second light source 111b is a semiconductor laser having a wavelength 795 nm (a second wavelength Δ2) and a maximum light output of 1 W. The receiving unit 120 is a linear array including piezoelectric elements having frequency characteristics of 4 MHz at the center frequency and 2 to 6 MHz at a 6-dB bandwidth. Between the receiving unit 120 and the object 100 is filled with an ultrasonic wave gel for acoustic matching.

In the present embodiment, complementary codes with a code length of 8 are used. Specifically, a first coding sequence {aᵢ}={1, 1, −1, 1, −1, −1, −1, 1}, a second coding sequence {bᵢ}={1, 1, −1, 1, 1, 1, 1, −1}, a third coding sequence {cᵢ}={1, −1, −1, −1, −1, 1, −1, −1}, and a fourth coding sequence {dᵢ}={1, −1, −1, −1, 1, −1, 1, 1} are used.

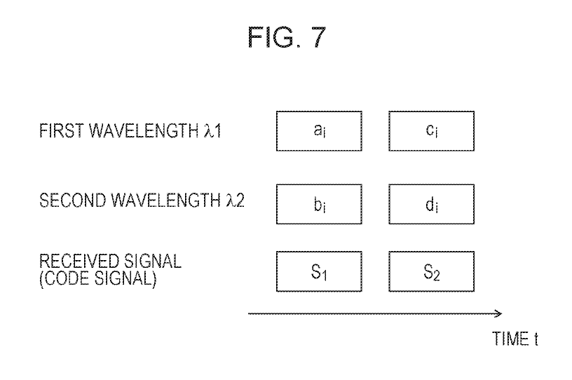

In the present embodiment, irradiation with intensity-modulated light of the first wavelength, irradiation with intensity-modulated light of the second wavelength, and acquisition of the received signals of photoacoustic waves generated by the irradiation with the intensity-modulated light are performed for encoding according to the sequence of FIG. 7.

First, the light irradiation unit 110 irradiates the object 100 with the intensity-modulated light of the first wavelength corresponding to the first coding sequence {aᵢ} and the intensity-modulated light of the second wavelength corresponding to the second coding sequence {bᵢ} in synchronism. The receiving unit 120 receives photoacoustic waves generated by the light irradiation and outputs a received signal S₁.

Next, the light irradiation unit 110 irradiates the object 100 with intensity-modulated light of the first wavelength corresponding to the third coding sequence {cᵢ} and intensity-modulated light of the second wavelength corresponding to the fourth coding sequence {dᵢ} in synchronism. The receiving unit 120 receives photoacoustic waves generated by the light irradiation and outputs a received signal S₂. The thus-obtained received signals S₁ and S₂ are coded signals (code signals) according to the present embodiment.

Encoding and decoding according to the present embodiment will be described in detail hereinbelow.

The control unit 153 transmits information on the first coding sequence {aᵢ} to the first driving unit 113a, and information on the second coding sequence {bᵢ} to the second driving unit 113b.

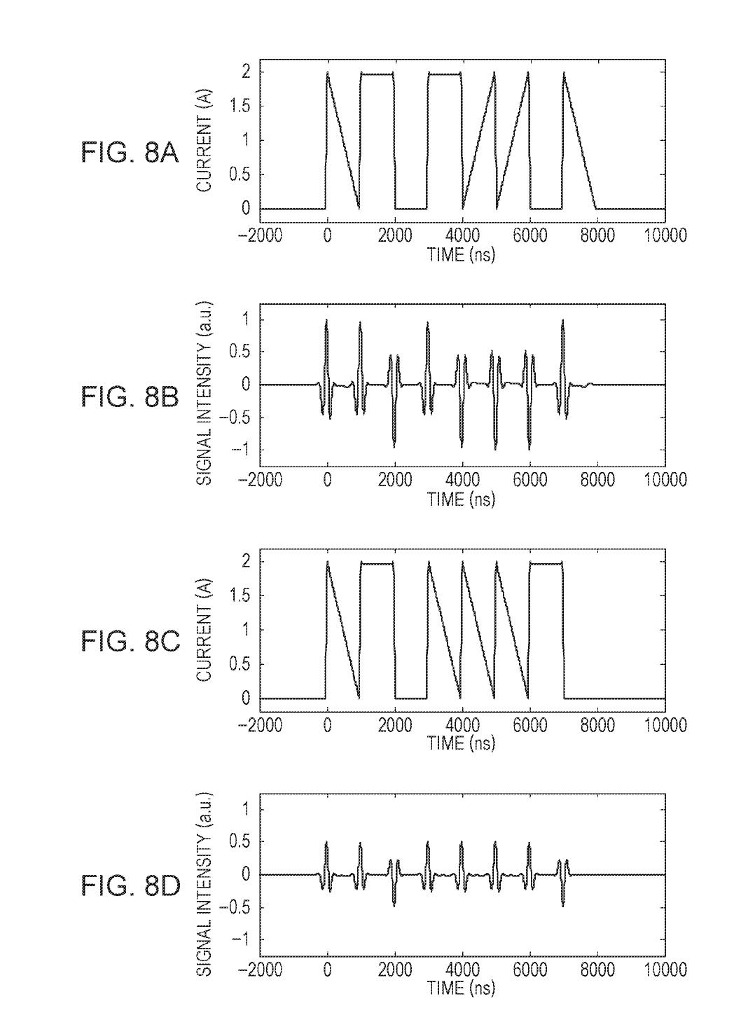

FIG. 8A illustrates the light output waveform (first light-modulated light of the first wavelength) of the first light source 111a driven by a driving current generated by the first driving unit 113a on the basis of information on the first coding sequence {aᵢ}. The time interval of the reference timing (corresponding to the cycle of the coding elements) is 1,000 ns. The rise time and the fall time of the first driving current corresponding to the positive coding element are 50 ns and 950 ns, respectively. The rise time and the fall time of the second driving current corresponding to the negative coding element are respectively 950 ns and 50 ns.

Assume that a point-like light absorber is present at a depth of 3 mm in the object 100.

Assume that the ratio of the absorption coefficient of the light absorber for light of a wavelength of 755 nm and light of a wavelength of 795 nm is 1:0.5.

A received signal obtained by receiving a photoacoustic wave generated when the light illustrated in FIG. 8A is applied to the point-like light absorber (sound source) with the receiving unit 120 has a waveform as illustrated in FIG. 8B. Actually, a time corresponding to the time the photoacoustic wave is propagated from the point-like light absorber to the receiving unit 120 (about 2 μs) shifts, but the time is ignored in this graph.

FIG. 8C illustrates the light output waveform (second light-modulated light of the second wavelength) of the second light source 111b driven by a driving current generated by the second driving unit 113b on the basis of information on the second coding sequence {bᵢ}. As in FIG. 8A, the time interval of the reference timing (corresponding to the cycle of the coding elements) is 1,000 ns. The rise time and the fall time of the first driving current corresponding to the positive coding element are 50 ns and 950 ns, respectively. The rise time and the fall time of the second driving current corresponding to the negative coding element are 950 ns and 50 ns, respectively.

A received signal obtained by receiving a photoacoustic wave generated when the light illustrated in FIG. 8C is applied to the point-like light absorber (sound source) with the receiving unit 120 has a waveform as illustrated in FIG. 8D. Actually, a time corresponding to the time the photoacoustic wave is propagated from the point-like light absorber to the receiving unit 120 (about 2 μs) shifts, but the time is ignored in this graph.

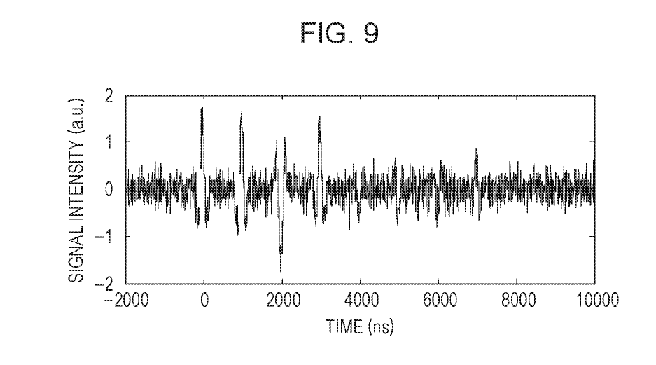

When the light output from the first light source 111a and the light output from the second light source 111b are synchronized (when the light is emitted at substantially the same timing), the received signal obtained by the receiving unit 120 is the sum of FIG. 8B and FIG. 8D. A received signal S₁(t) obtained by performing irradiation with the first intensity-modulated light of the first wavelength and the irradiation with the second intensity-modulated light of the second wavelength at substantially the same timing is illustrated in FIG. 9. Here, to describe a noise reduction effect, noise with a mean value of 0 and a standard deviation of 0.2 is added.

Subsequently, the control unit 153 transmits information on the third coding sequence {cᵢ} to the first driving unit 113a, and information on the fourth coding sequence {dᵢ} to the second driving unit 113b.

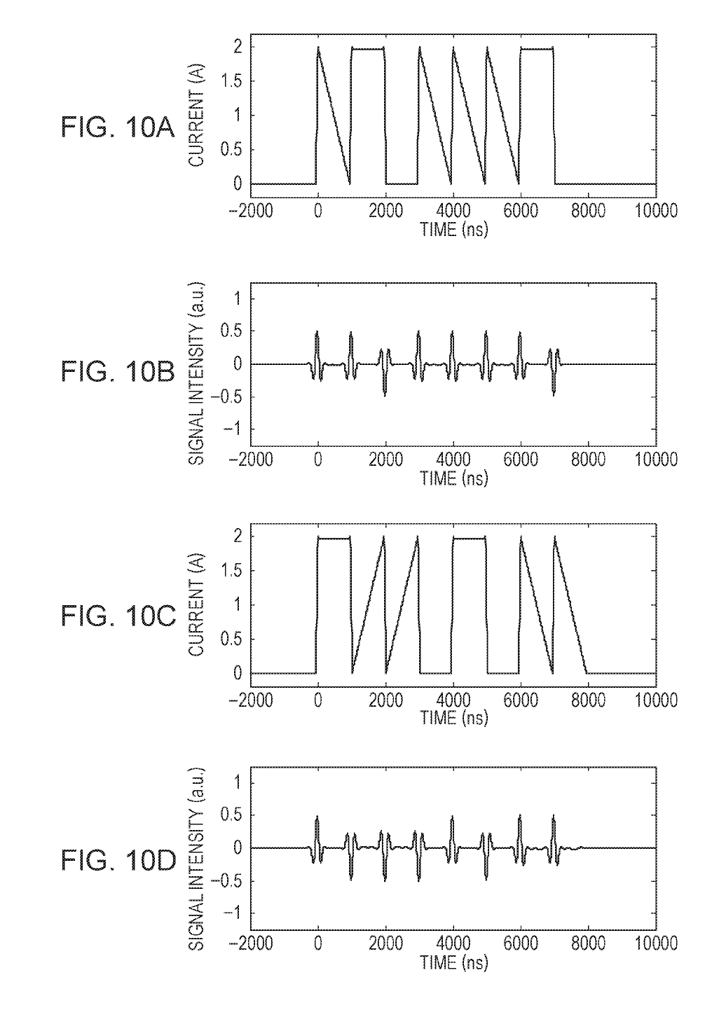

FIG. 10A illustrates the light output waveform (third light-modulated light of the first wavelength) of the first light source 111a driven by a driving current generated by the first driving unit 113a on the basis of information on the third coding sequence {cᵢ}. As in FIG. 8A, the time interval of the reference timing (corresponding to the cycle of the coding elements) is 1,000 ns. The rise time and the fall time of the first driving current corresponding to the positive coding element are 50 ns and 950 ns, respectively. The rise time and the fall time of the second driving current corresponding to the negative coding element are 950 ns and 50 ns, respectively.

A received signal obtained by receiving a photoacoustic wave generated when the light illustrated in FIG. 10A is applied to the point-like light absorber (sound source) with the receiving unit 120 has a waveform as illustrated in FIG. 10B. Actually, a time corresponding to the time the photoacoustic wave is propagated from the point-like light absorber to the receiving unit 120 (about 2 μs) shifts, but the time is ignored in this graph.

FIG. 10C illustrates the light output waveform (fourth light-modulated light of the second wavelength) of the second light source 111b driven by a driving current generated by the second driving unit 113b on the basis of information on the fourth coding sequence {dᵢ}. As in FIG. 8A, the time interval of the reference timing (corresponding to the cycle of the coding elements) is 1,000 ns. The rise time and the fall time of the first driving current corresponding to the positive coding element are 50 ns and 950 ns, respectively. The rise time and the fall time of the second driving current corresponding to the negative coding element are 950 ns and 50 ns, respectively.

A received signal obtained by receiving a photoacoustic wave generated when the light illustrated in FIG. 10C is applied to the point-like light absorber (sound source) with the receiving unit 120 has a waveform as illustrated in FIG. 10D. Actually, a time corresponding to the time the photoacoustic wave is propagated from the point-like light absorber to the receiving unit 120 (about 2 μs) shifts, but the time is ignored in this graph.

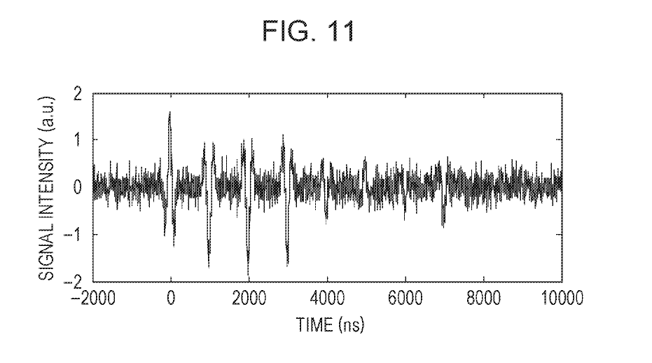

When the light output from the first light source 111a and the light output from the second light source 111b are synchronized, the received signal obtained by the receiving unit 120 is the sum of FIG. 10B and FIG. 10D. A received signal S₂(t) obtained by performing irradiation with the third intensity-modulated light of the first wavelength and the irradiation with the fourth intensity-modulated light of the second wavelength at substantially the same timing is illustrated in FIG. 11. Here, to describe a noise reduction effect, noise with a mean value of 0 and a standard deviation of 0.2 is added.

A method for decoding a coded received signal performed by the operating unit 151 in the computer 150 will be described.

The operating unit 151 performs a decoding process according to Expression 3 on the received signals S1 and S₂ to obtain a decoded signal DS₁(t) corresponding to the first wavelength. The operating unit 151 performs a decoding process according to Expression 4 on the received signals S1 and S₂ to obtain a decoded signal DS₂(t) corresponding to the second wavelength.

$$\begin{matrix}[ Math . 3 ] & \\ {DS}_{1} ( t ) = {\sum }_{i = 1}^{N} {S}_{1} ( t + ( i - 1 ) Δ t ) \cdot {a}_{i} + {\sum }_{i = 1}^{N} {S}_{2} ( t + ( i - 1 ) Δ t ) \cdot {c}_{i} & ( 3 ) \\ [ Math . 4 ] & \\ {DS}_{2} ( t ) = {\sum }_{i = 1}^{N} {S}_{1} ( t + ( i - 1 ) Δ t ) \cdot {b}_{i} + {\sum }_{i = 1}^{N} {S}_{2} ( t + ( i - 1 ) Δ t ) \cdot {d}_{i} & ( 4 )\end{matrix}$$

where Δt is the time interval of the reference timing.

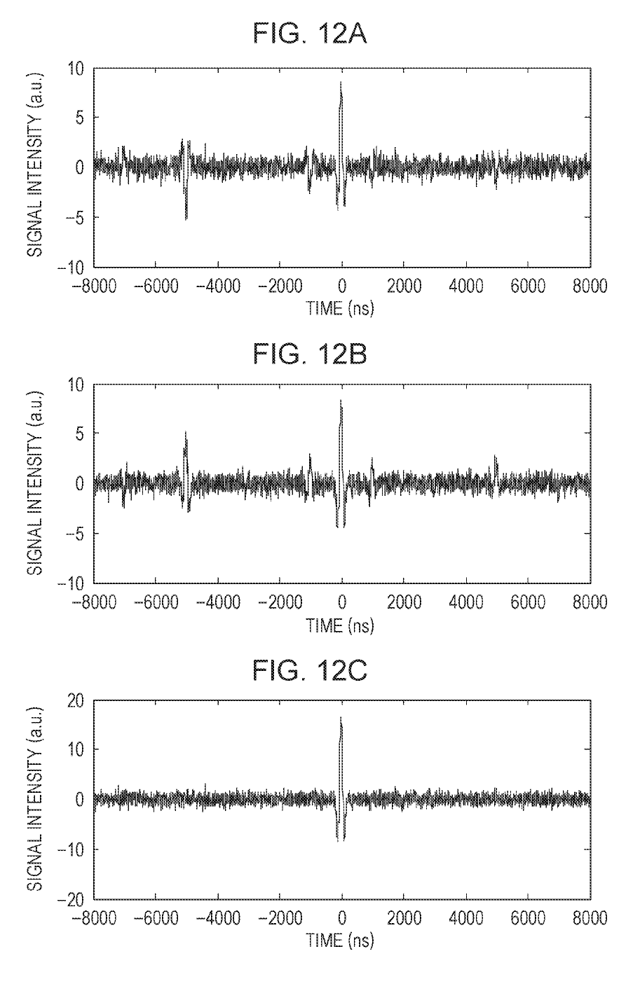

As a result of the decoding process of the first term on the right side of Expression 3 on the reception waveform (the received signal S₁ obtained from the first intensity-modulated light of the first wavelength and the second intensity-modulated light of the second wavelength) in FIG. 9, the decoded received signal as in FIG. 12A can be obtained. As a result of the decoding process of the second term on the right side of Expression 3 on the reception waveform (the received signal S₂ obtained from the third intensity-modulated light of the first wavelength and the fourth intensity-modulated light of the second wavelength) in FIG. 11, the decoded received signal as in FIG. 12B can be obtained. The sum of the signal in FIG. 12A and the signal in FIG. 12B forms the waveform (a decoded signal DS₁) in FIG. 12C. In other words, the peak value of the received signal DS₁ decoded according to Expression 3 is 16 times the peak value of a received signal obtained by single light irradiation, and the sidelobes are suppressed below noise.

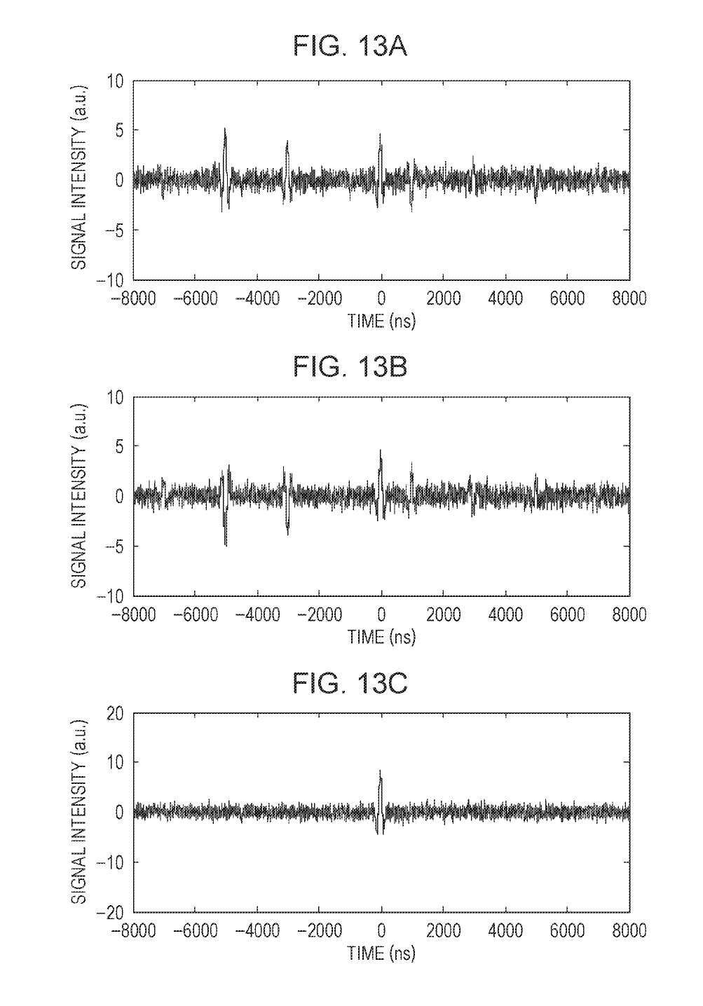

As a result of the decoding process of the first term on the right side of Expression 4 on the reception waveform (the received signal S₁ obtained from the first intensity-modulated light of the first wavelength and the second intensity-modulated light of the second wavelength) in FIG. 9, the decoded received signal as in FIG. 13A can be obtained. As a result of the decoding process of the second term on the right side of Expression 4 on the reception waveform (the received signal S₂ obtained from the third intensity-modulated light of the first wavelength and the fourth intensity-modulated light of the second wavelength) in FIG. 11, the decoded received signal as in FIG. 13B can be obtained. The sum of the signal in FIG. 13A and the signal in FIG. 13B forms the waveform (a decoded signal DS₂) in FIG. 13C. In other words, the peak value of the received signal DS₂ decoded according to Expression 4 is 16 times the peak value of a received signal obtained by single light irradiation, and the sidelobes are suppressed below noise.

A comparison between FIG. 12C and FIG. 13C shows that the ratio of the absorption coefficient of the light absorber is stored. This indicates that information on the light absorption of the object, included in the received signal, is kept stored even after encoding and decoding. This enables spectral information, such as the hemoglobin concentration of blood, to be acquired by analyzing the decoded signals DS₁ and DS₂ corresponding to the individual wavelengths.

The comparison also shows that the noise levels of the signals illustrated in FIG. 12C and FIG. 13C are respectively lower than the noise levels of the signals illustrated in FIG. 9 and FIG. 11. The use of the set of coding sequences having a code length of 8 as in the present embodiment increases the signal level by 16 times, the noise level by four times, improving the S/N ratio by four times.

The operating unit 151 can obtain a photographic image in which the signal-to-noise (S/N) ratio is improved by using the thus-obtained decoded signal. If the receiving unit 120 includes a plurality of transducers, a received signal output from each transducer is decoded, so that a decoded signal is generated for each transducer. The operating unit 151 can generate a photographic image using a plurality of decoded signals corresponding to the plurality of transducers with the reconstruction method described above or the like.

In the present embodiment, light irradiation (negative intensity-modulated light) corresponding to the negative coding element is performed at the time of encoding by light irradiation. This allows high-accuracy encoding on the basis of a coding sequence including a negative coding element. Thus, the present embodiment allows such a coded signal to be decoded with high accuracy by decoding based on a coding sequence containing a negative coding element (for example, the decoding process illustrated in Expressions 3 and 4). In this manner, the light irradiation corresponding to a negative coding element allows higher-accuracy decoding than that without light irradiation corresponding to a negative coding element.

In the present embodiment, simultaneous reception time is achieved by irradiating the object with the irradiation light from the first light source 111a and the irradiation light from the second light source 111b at substantially the same timing. This improves the S/N ratio in a shorter time than a case in which photoacoustic waves from the lights of two different wavelengths are separately received.

In the present embodiment, lights of two wavelengths are synchronously applied to the object, but it is not absolutely necessary to apply lights at the same timing. To decrease the measuring time, lights may be applied in such a manner that at least part of the irradiation periods overlap between the wavelengths (at least part of the reception times is overlapped between the wavelengths).

In the case of temporally separately receiving photoacoustic waves from lights of two wavelengths, if the object moves during that time, a time lag occurs between the signals. However, the method of the present embodiment can reduce the time lag between the signals when the object moves by overlapping the light irradiation time between the lights of different wavelengths.

The upper limit of the time interval of the reference timing of coding elements according to the present embodiment will be described.

The present embodiment uses two code sequences with a code length of 8 in one wavelength, so that the signal level of a decoded received signal is 16 times, and the noise level is four times. This increases the S/N ratio by four times.

To achieve the same S/N ratio with a generally known method for generating photoacoustic waves by applying impulse light, it is necessary to simply obtain a received signal 16 times and average the obtained signals. The time required to obtain a received signal 16 times by the general method is 16T_{tof}. Furthermore, it is necessary to obtain received signals of two wavelengths, so that the time required for measurement is 32T_{tof}.

In the present embodiment, the time required to obtain a received signal corresponding to the coding sequence {aᵢ} is the sum of the time during which light corresponding to the coding sequence {aᵢ} and the time until a photoacoustic wave generated by light corresponding to the last coding element reaches the receiving unit. In other words, the time is expressed as 7Δt+T_{tof}. The times required to obtain received signals corresponding to the second to fourth coding sequences {bᵢ}, {cᵢ}, and {dᵢ} are also the same. Therefore, when received signals corresponding to the first to fourth coding sequences {aᵢ}, {bᵢ}, {cᵢ}, and {dᵢ} are acquired in sequence (in series), the time required to obtain received signals based on the first and second wavelengths is 28Δt+T_{tof}.

In the present embodiment, the light of the first wavelength and light of the second wavelength are applied at the same time to acquire the received signals at the same time. This reduces the time required to obtain received signals according to the present embodiment to 14Δt+2T_{tof}, thus reducing the time required to obtain received signals as compared with the method of temporally separating signals of different wavelengths.

It can be said that the method according to the present embodiment has a higher effect of improving the S/N ratio than that of the general method on the condition that the time required to obtain received signals is shorter. This condition is expressed as 14Δt<30T_{tof}. This condition generalized using a code length N is as follows:

$$\begin{matrix}[ Math . 5 ] & \\ Δ t < \frac{2 N - 1}{N - 1} {T}_{tof} & ( 5 )\end{matrix}$$

It may be that, if N is great to some extent, Δt<2T_{tof} hold. In other words, it may be that the time interval of the reference timing be shorter than two times the time required for photoacoustic waves generated at the deepest portion of the observation region of the object to reach the receiving unit. For example, when the deepest portion of the observation region of the object is 5 cm deep, and the sound velocity in the object is 1,500 m/s, the time required for the photoacoustic waves generated at the deepest portion to reach the receiving unit is 33 μs. In this case, the time interval of the reference timing is preferably shorter than 66 μs. In the case of a code length of 8, the time interval is preferably shorter than 71 μs from Expression 5. The control unit 153 may change the time interval of the reference timing according to the region of interest that the user indicates using the input unit 170 so that the time interval is shorter than the time until the photoacoustic wave generated in the deepest part reaches the receiving unit. Alternatively, the control unit 153 may change the time interval of the reference timing according to the instruction of the user or the sound velocity of the object determined by calculation, so that the time interval is shorter than the time until the photoacoustic wave generated in the deepest part reaches the receiving unit. The deepest part is an observation region (the region of interest) farthest from the receiving unit.

### [Configuration of Driving Unit]

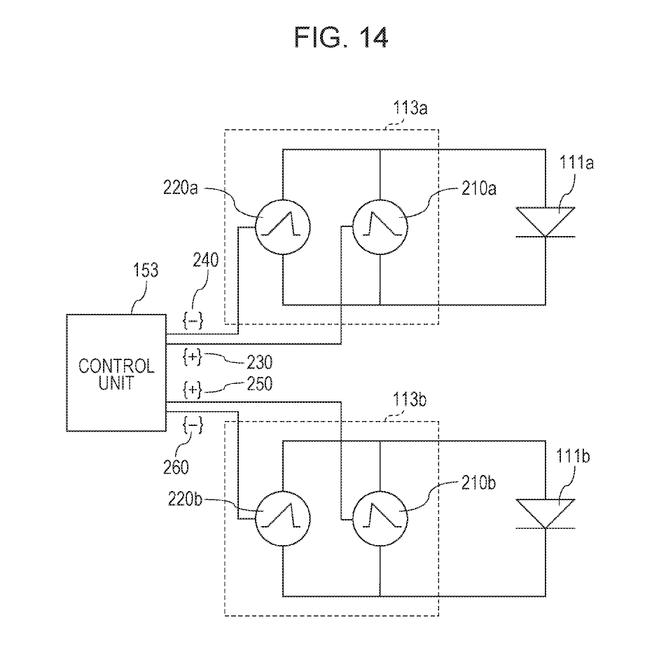

The first driving unit 113a or the second driving unit 113b may include one power source capable of generating both of the first driving current and the second driving current. Alternatively, the first driving unit 113a or the second driving unit 113b may include a first power source capable of generating the first driving current and a second power source capable of generating the second driving current. Here, an example in which the driving currents are generated from separate power sources will be described with reference to FIG. 14.

The first driving unit 113a illustrated in FIG. 14 includes a first power source 210a capable of generating the first driving current and a second power source 220a capable of generating the second driving current. The control unit 153 has the function of transmitting a first control signal 230 formed of 1 and 0 and a second control signal 240 formed of −1 and 0 to the first driving unit 113a.

For example, for light irradiation corresponding to the first coding sequence {aᵢ}={1, 1, −1, 1, −1, −1, −1, 1}, the control unit 153 separates a control signal into the first control signal 230 {1, 1, 0, 1, 0, 0, 0, 1} and the second control signal 240 {0, 0, −1, 0, −1, −1, −1, 0} and transmits the control signals 230 and 240 to the first driving unit 113a. In other words, the control unit 153 transmits the first control signal 230 to the first power source 210a and the second control signal 240 to the second power source 220a.

The first power source 210a generates the first driving current according to the timing of the coding element {1} of the first control signal 230 and generates no electric current or an electric current in which generation of a photoacoustic wave is suppressed at the timing of the coding element {0} of the first control signal 230. The second power source 220a generates the second driving current according to the timing of the coding element {−1} of the second control signal 240 and generates no electric current or an electric current in which generation of a photoacoustic wave is suppressed at the timing of the coding element {0} of the second control signal 240. As a result, an electric current similar to the driving current (FIG. 8A) corresponding to the first coding sequence {aᵢ} is input to the first light source 111a.

The second driving unit 113b illustrated in FIG. 14 includes a third power source 210b capable of generating the first driving current and a fourth power source 220b capable of generating the second driving current. The control unit 153 has the function of transmitting a third control signal 250 formed of 1 and 0 and a fourth control signal 260 formed of −1 and 0 to the second driving unit 113b.

For example, for light irradiation corresponding to the second coding sequence {bᵢ}={1, 1, −1, 1, 1, 1, 1, −1}, the control unit 153 separates a control signal into the third control signal 250 {1, 1, 0, 1, 0, 1, 1, 1} and the fourth control signal 260 {0, 0, −1, 0, 0, 0, 0, −1} and transmits the control signals 250 and 260 to the second driving unit 113b. In other words, the control unit 153 transmits the third control signal 250 to the third power source 210b and the fourth control signal 260 to the fourth power source 220b.

The third power source 210b generates the first driving current according to the timing of the coding element {1} of the third control signal 250 and generates no electric current or an electric current in which generation of a photoacoustic wave is suppressed at the timing of the coding element {0} of the third control signal 250. The fourth power source 220b generates the second driving current according to the timing of the coding element {−1} of the fourth control signal 260 and generates no electric current or an electric current in which generation of a photoacoustic wave is suppressed at the timing of the coding element {0} of the fourth control signal 260. As a result, an electric current similar to the driving current (FIG. 8C) corresponding to the third coding sequence {bᵢ} is input to the second light source 111b.

An apparatus that uses a different power source for each driving current can be simplified in the design of the first driving unit 113a or the second driving unit 113b as compared with an apparatus that generates different driving currents with a single power source. Using a different power source for each driving current offers high responsivity in high-speed switching between different driving currents. Using a different power source for each driving current when a light source is provided for each driving current allows different driving currents to be input to the separate light sources during the same period. This allows lights with different coding elements to be applied to the object in a temporally overlapping manner. This increases the light irradiation efficiency, allowing decoded signals with high a S/N ratio to be obtained in a short time.

In the present embodiment, the maximum intensities of the peak light output of the first light source 111a and the second light source 111b are equal. However, the present disclosure is not limited thereto. In the present embodiment, the level of {1} and the level of {−1} of the first wavelength need to be equal to some extent, and the level of {1} and the level of {−1} of the second wavelength also need to be equal to some extent, where “to some extent” means that variations in the level are averaged to a negligible level. However, the level of {1} of the first wavelength and the level of {1} of the second wavelength do not need to be equal. For example, depending on the individual differences of the light sources, light output can differ even with the same input current. In that case, the input current may be changed for each wavelength so that the maximum intensities of the light outputs match. Even if the maximum intensities of the peak light outputs differ, the difference can be corrected by standardizing the decoded received signals to the maximum peak intensities of the respective light outputs. Alternatively, after the received signals are standardized to the maximum peak intensities of the respective light outputs, the decoding process may be performed.

The code length and the time interval of the reference timing in the present embodiment are not limited to the above. They may be selected as appropriate according to the depth of the observation region in the object or the performance of the light source driving unit so that the S/N ratio can be improved. In the present embodiment, an example of acquiring decoded signals corresponding to two wavelengths has been described, but it is only required to acquire a decoded signal corresponding to at least one of the two wavelengths. That is, in the present embodiment, it is only required to acquire a decoded signal corresponding to at least one of the plurality of wavelengths. This also allows acquisition of a decoded signal corresponding to a desired wavelength.

### Second Embodiment

### [Encoding and Decoding Using Orthogonal Codes]

Although two sets of complementary codes that satisfy “complete orthogonal relationship” are present, three or more pairs of complementary codes that satisfy “complete orthogonal relationship” one another are not present. Therefore, the method described in the first embodiment cannot be applied to lights of three or more wavelengths.

A method of encoding and decoding using lights of three or more wavelengths will be described below.

Consider four coding sequences {aᵢ^{k}}(k=1 to 4, i=1 to 4) orthogonal to one another and having a code length of 4:

a first coding sequence {aᵢ¹}={1, −1, −1, 1}
a second coding sequence {aᵢ²}={1, −1, 1, −1}
a third coding sequence {aᵢ³}={1, 1, −1, −1}
a fourth coding sequence {aᵢ⁴}={1, 1, 1, 1}
Furthermore, consider four different permutations {gₘ}(m=1 to 4, each element is one of 1 to 4) indicating the order for arranging these four coding sequences in order without overlapping:
a first permutation {g₁}={{aᵢ²}, {aᵢ³}, {aᵢ⁴}}
a second permutation {g₂}={{aᵢ²}, {aᵢ⁴}, {aᵢ³}}
a third permutation {g₃}={{aᵢ³}, {aᵢ⁴}, {aᵢ²}}
a fourth permutation {g₄}={{aᵢ⁴}, {aᵢ³}, {aᵢ²}}

Consider the total sum of the respective cross-correlation functions of the four coding sequences arranged according to the permutations gₚ and g_{q}. The total sum can be expressed as follows:

$$\begin{matrix}[ Math . 6 ] & \\ {\sum }_{j = 1}^{4} ( {a}^{{g}_{p} ( j )} * {a}^{{g}_{q} ( j )} ) & ( 6 )\end{matrix}$$

(p and q are integers from 1 to 4)

If p=q, Expression 6 takes the total sum of the autocorrelation functions. The total sum is 16 at the peak, and O at all points other than the peak. That is,

(a¹* a¹)={1, −2, −1, 4, −1, −2, 1}
(a²* a²)={−1, 2, −3, 4, −3, 2, −1}
(a³* a³)={−1, −2, 1, 4, 1, −2, −1}
(a⁴* a⁴)={1, 2, 3, 4, 3, 2, 1}
and, at any p,

$${\sum }_{j = 1}^{4} ( {a}^{{g}_{p} ( j )} * {a}^{{g}_{p} ( j )} ) = ( {a}^{1} * {a}^{1} ) + ( {a}^{2} * {a}^{2} ) + ( {a}^{3} * {a}^{3} ) + ( {a}^{4} * {a}^{4} ) = \{ 0 , 0 , 0 , 16 , 0 , 0 , 0 \} \qquad [ Math . 7 ]$$

holds.

If p≠q, Expression 6 takes the total sum of the autocorrelation functions. The total sum is 0 in all combinations of p≠q. For example, if p=1 and q=2,

(a¹* a²)={−1, 2, −1, 0, 1, −2, 1}
(a²* a¹)={1, −2, 1, 0, −1, 2, −1}
(a³* a⁴)={1, 2, 1, 0, −1, −2, −1}
(a⁴* a³)={−1, −2, −1, 0, 1, 2, 1}

$${\sum }_{j = 1}^{4} ( {a}^{{g}_{1} ( j )} * {a}^{{g}_{2} ( j )} ) = ( {a}^{1} * {a}^{2} ) + ( {a}^{2} * {a}^{1} ) + ( {a}^{3} * {a}^{4} ) + ( {a}^{4} * {a}^{3} ) = \{ 0 , 0 , 0 , 0 , 0 , 0 , 0 \} \qquad [ Math . 8 ]$$

holds.

Using a set of coding sequences having such characteristics allows the following to be achieved.
When signals encoded according to four coding sequences determined by a permutation gₚ are sequentially obtained, and the obtained signals are sequentially decoded according to four coding sequences determined by the same permutation gₚ, the total sum of the decoded signals is a delta function.
When signals encoded according to four coding sequences determined by a permutation gₚ are sequentially obtained, and the obtained signals are sequentially decoded according to four coding sequences determined by a different permutation g_{q}, the total sum of the decoded signals is zero.

Using such coding sequences and permutations indicating their orders in a photoacoustic imaging apparatus that uses lights of a plurality of different wavelengths allows a signal obtained from light of a certain wavelength and a signal obtained from light of the other wavelengths to be separately obtained even if the light irradiation times overlap.

The relationship among the number of wavelengths, code lengths, and the number of coding sequences orthogonal to each other will be described. When the number of wavelengths is from two to four, it may be to set the code length to 4, and the number of orthogonal codes to 4. When the number of wavelengths is from 5 to 8, it may be to set the code length to 8, and the number of orthogonal codes to 8. It may be to set the number of orthogonal coding sequences to the power of 2 equal to or greater than the number of wavelengths.

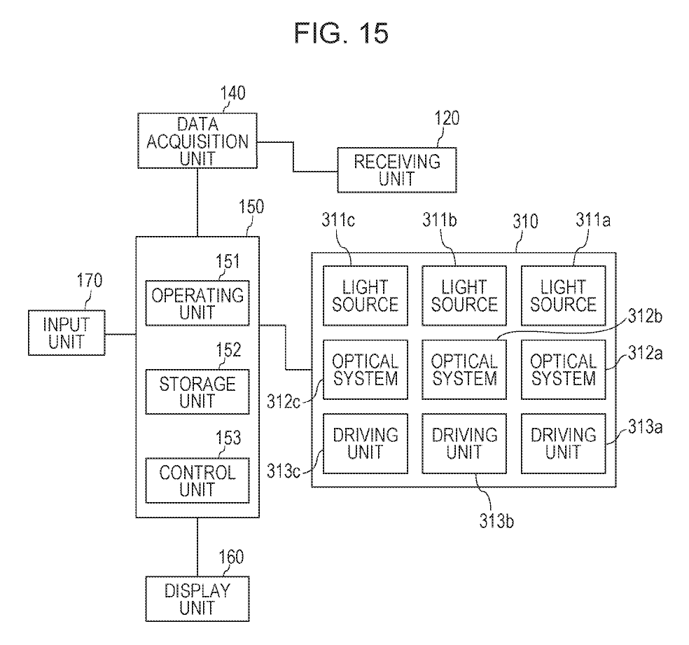

In the present embodiment, a case in which lights of three different wavelengths are used as irradiation light in the photoacoustic imaging apparatus illustrated in FIG. 15 will be described. Components common to FIG. 3 are given the same reference signs, and descriptions thereof will be omitted. In the present embodiment, a semiconductor laser with a wavelength 755 nm (first wavelength Δ1) and a maximum light output of 1 W is used as a first light source 311a. A semiconductor laser with a wavelength 795 nm (a second wavelength Δ2) and a maximum light output of 1 W is used as a second light source 311b. A semiconductor laser with a wavelength 825 nm (a third wavelength Δ3) and a maximum light output 1 W is used as a third light source 311c. The receiving unit 120 is a linear array including piezoelectric elements having frequency characteristics of a center frequency of 4 MHz and frequencies of 2 to 6 MHz in a 6-dB band. Between the receiving unit 120 and the object 100 is filled with an ultrasonic wave gel for acoustic matching.

In the present embodiment, four coding sequences orthogonal to one another and having a code length of 4 are used. Specifically,

a first coding sequence {aᵢ¹}={1, −1, −1, 1}
a second coding sequence {aᵢ²}={1, −1, 1, −1}
a third coding sequence {aᵢ³}={1, 1, −1, −1}
a fourth coding sequence {aᵢ⁴}={1, 1, 1, 1}
are used. Permutations assigned to the lights of the first, second, and third wavelengths are a first permutation {g₁}={{aᵢ²}, {aᵢ³}, {a⁴}}, a second permutation {g₂}={{aᵢ²}, {aᵢ⁴}, {aᵢ³}}, and a third permutation {g₃}={{aᵢ³}, {aᵢ⁴}, {aᵢ¹}, {aᵢ²}}.

In other words, the four coding sequences are assigned to the light of the first wavelength in the order determined by the first permutation. The four coding sequences are assigned to the light of the second wavelength in the order determined by the second permutation. The four coding sequences are assigned to the light of the third wavelength in the order determined by the third permutation.

A process of encoding and decoding of the photoacoustic imaging apparatus according to the present embodiment will be described. Assume that a point-like light absorber is present at a depth of 3 mm in the object 100. Assume that the ratio of the absorption coefficient of the light absorber for light of a wavelength 755 nm, light of a wavelength 795 nm, and the light of a wavelength 825 nm is 1:0.5:0.75.

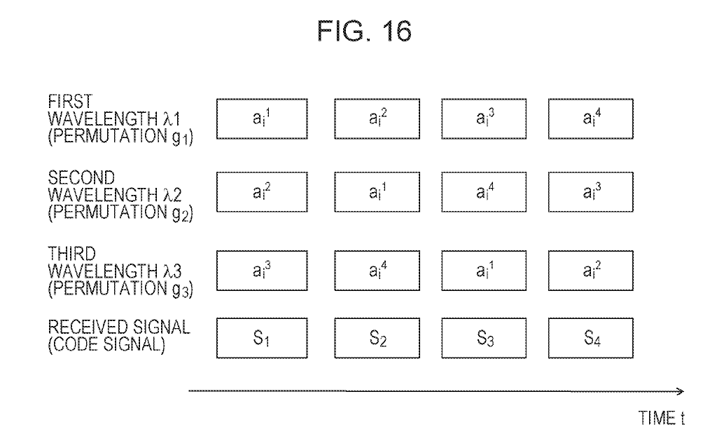

In the present embodiment, intensity-modulated lights with elements of the same number of the permutation are synchronously applied to the object 100 in the sequence of FIG. 16 for encoding.

First, for the first permutation element, the light irradiation unit 110 synchronously applies intensity-modulated light of the first wavelength corresponding to the first coding sequence {aᵢ¹}, intensity-modulated light of the second wavelength corresponding to the second coding sequence {aᵢ²}, and intensity-modulated light of the third wavelength corresponding to the third coding sequence {aᵢ³}. The receiving unit 120 receives photoacoustic waves generated by the light irradiation and outputs a received signal S1.

Next, for the second permutation element, the light irradiation unit 110 synchronously applies intensity-modulated light of the first wavelength corresponding to the second coding sequence {aᵢ²}, intensity-modulated light of the second wavelength corresponding to the first coding sequence {aᵢ¹}, and intensity-modulated light of the third wavelength corresponding to the fourth coding sequence {aᵢ⁴}. The receiving unit 120 receives photoacoustic waves generated by the light irradiation and outputs a received signal S₂.

Next, for the third permutation element, the light irradiation unit 110 synchronously applies intensity-modulated light of the first wavelength corresponding to the third coding sequence {aᵢ³}, intensity-modulated light of the second wavelength corresponding to the fourth coding sequence {aᵢ⁴}, and intensity-modulated light of the third wavelength corresponding to the first coding sequence {aᵢ¹}. The receiving unit 120 receives photoacoustic waves generated by the light irradiation and outputs a received signal S3.

Next, for the fourth permutation element, the light irradiation unit 110 synchronously applies intensity-modulated light of the first wavelength corresponding to the fourth coding sequence {aᵢ⁴}, intensity-modulated light of the second wavelength corresponding to the third coding sequence {aᵢ³}, and intensity-modulated light of the third wavelength corresponding to the second coding sequence {aᵢ²}. The receiving unit 120 receives photoacoustic waves generated by the light irradiation and outputs a received signal S4.

The irradiation with the lights of the individual wavelengths of each permutation element may not be synchronized completely. However, at least part of the irradiation periods of the intensity-modulated lights of the plurality of wavelengths may be overlapped to improve the S/N ratio of the signals obtained per unit time.

### [S1. Application of Intensity-Modulated Light Corresponding to First Permutation Element]

The control unit 153 transmits information on the first coding sequence {aᵢ¹} to a first driving unit 313a according to the assigned permutation. The control unit 153 transmits information on the second coding sequence {aᵢ²} to a second driving unit 313b according to the assigned permutation. The control unit 153 transmits information on the third coding sequence {aᵢ³} to a third driving unit 313c according to the assigned permutation.

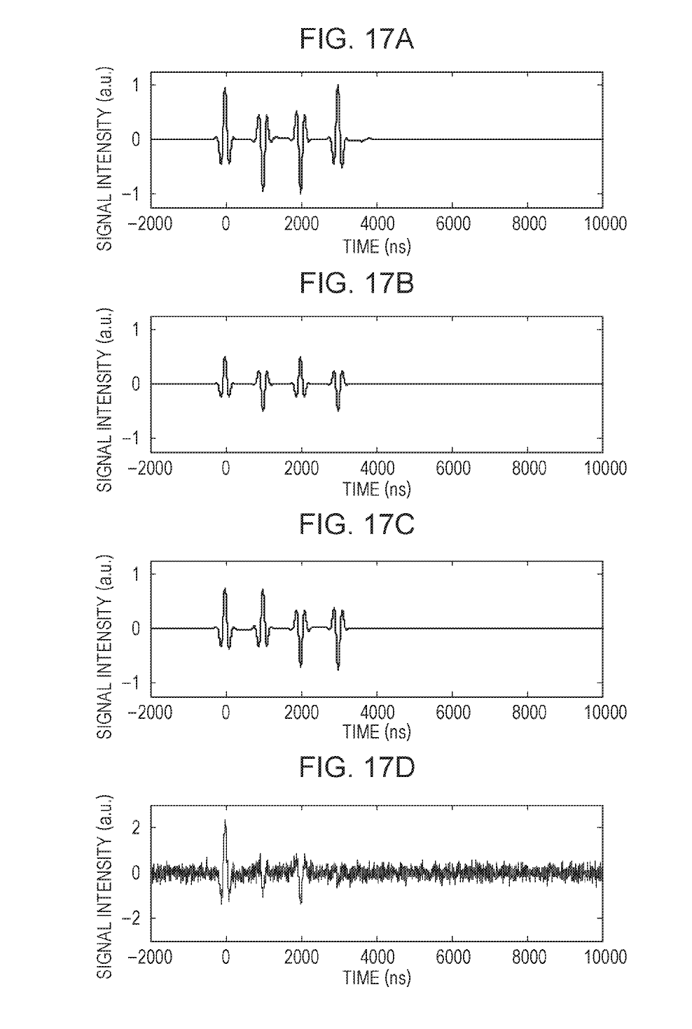

The first light source 311a is driven by a driving current generated by the first driving unit 313a according to the information on the first coding sequence {aᵢ¹}. The generated light is applied to a point-like light absorber (sound source) via a first optical system 312a. A received signal obtained by receiving photoacoustic waves generated at that time with the receiving unit 120 has a waveform as illustrated in FIG. 17A.

The second light source 311b is driven by a driving current generated by the second driving unit 313b according to the information on the second coding sequence {aᵢ²}. The generated light is applied to the point-like light absorber (sound source) via a second optical system 312b. A received signal obtained by receiving photoacoustic waves generated at that time with the receiving unit 120 has a waveform as illustrated in FIG. 17B.

The third light source 311c is driven by a driving current generated by the third driving unit 313c according to the information on the third coding sequence {aᵢ³}. The generated light is applied to the point-like light absorber (sound source) via a third optical system 312c. A received signal obtained by receiving photoacoustic waves generated at that time with the receiving unit 120 has a waveform as illustrated in FIG. 17C.

### [S2. Acquisition of Received Signal Corresponding to First Permutation Element]

When the light outputs from the first light source 311a, the second light source 311b, and third light source 311c are synchronized (when the lights are emitted at substantially the same timing), the signal received by the receiving unit 120 is the sum of the signals illustrated in FIGS. 17A, 17B, and 17C. The received signal S₁(t) obtained at that time is illustrated in FIG. 17D. In other words, the signal S₁(t) is obtained by applying intensity-modulated light of the first wavelength corresponding to the first coding sequence {aᵢ¹}, intensity-modulated light of the second wavelength corresponding to the second coding sequence {aᵢ²}, and intensity-modulated light of the third wavelength corresponding to the third coding sequence {aᵢ³} at substantially the same timing. To describe the effect of reducing noise, noise of a mean value of 0 and a standard deviation of 0.2 is added.

### [S3. Application of Intensity-Modulated Light Corresponding to Second Permutation Element]

The control unit 153 transmits the information on the second coding sequence {aᵢ²} to the first driving unit 313a according to the assigned permutation. The control unit 153 transmits the information on the first coding sequence {aᵢ¹} to the second driving unit 313b according to the assigned permutation. The control unit 153 transmits the information on the fourth coding sequence {aᵢ⁴} to the third driving unit 313c according to the assigned permutation.

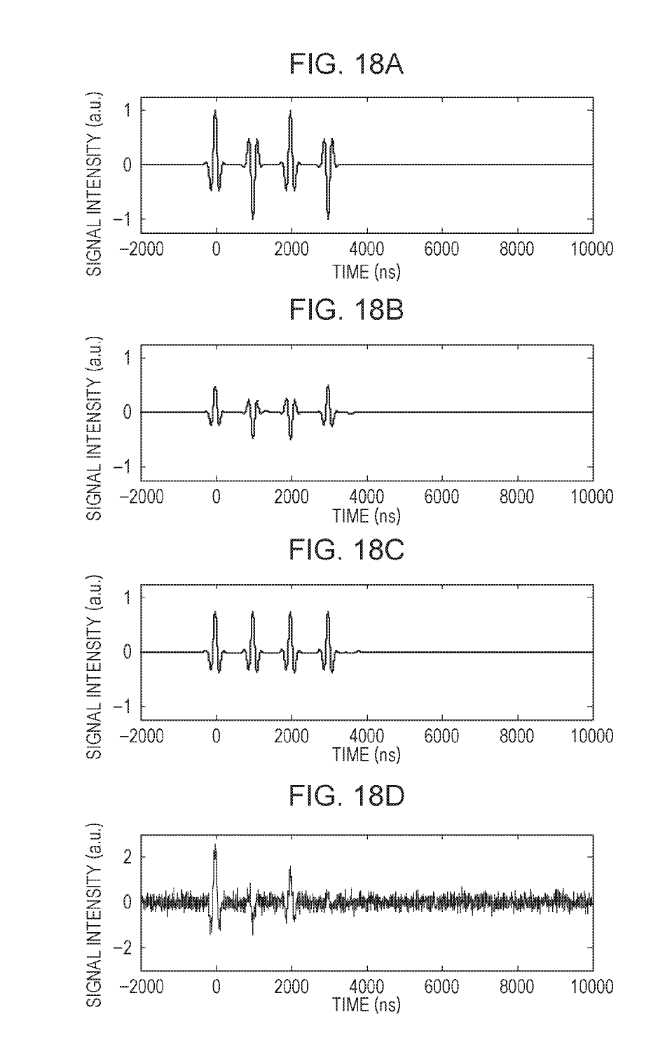

The first light source 311a is driven by a driving current generated by the first driving unit 313a according to the information on the second coding sequence {aᵢ²}. The generated light is applied to the point-like light absorber (sound source) via the first optical system 312a. A received signal obtained by receiving photoacoustic waves generated at that time with the receiving unit 120 has a waveform as illustrated in FIG. 18A.

The second light source 311b is driven by a driving current generated by the second driving unit 313b according to the information on the first coding sequence {aᵢ¹}. The generated light is applied to the point-like light absorber (sound source) via the second optical system 312b. A received signal obtained by receiving photoacoustic waves generated at that time with the receiving unit 120 has a waveform as illustrated in FIG. 18B.

The third light source 311c is driven by a driving current generated by the third driving unit 313c according to the information on the fourth coding sequence {aᵢ⁴}. The generated light is applied to the point-like light absorber (sound source) via the third optical system 312c. A received signal obtained by receiving photoacoustic waves generated at that time with the receiving unit 120 has a waveform as illustrated in FIG. 18C.

### [S4. Acquisition of Received Signal Corresponding to Second Permutation Element]

When the light outputs from the first light source 311a, the second light source 311b, and third light source 311c are synchronized (when the lights are emitted at substantially the same timing), the signal received by the receiving unit 120 is the sum of the signals illustrated in FIGS. 18A, 18B, and 18C. The received signal S₂(t) obtained at that time is illustrated in FIG. 18D. In other words, the signal S₂(t) is obtained by applying intensity-modulated light of the first wavelength corresponding to the second coding sequence {aᵢ²}, intensity-modulated light of the second wavelength corresponding to the first coding sequence {aᵢ¹}, and intensity-modulated light of the third wavelength corresponding to the fourth coding sequence {aᵢ⁴} at substantially the same timing. To describe the effect of reducing noise, noise of a mean value of 0 and a standard deviation of 0.2 is added.

### [S5. Application of Intensity-Modulated Light Corresponding to Third Permutation Element]

The control unit 153 transmits the information on the third coding sequence {aᵢ³} to the first driving unit 313a according to the assigned permutation. The control unit 153 transmits the information on the fourth coding sequence {aᵢ⁴} to the second driving unit 313b according to the assigned permutation. The control unit 153 transmits the information on the first coding sequence {aᵢ¹} to the third driving unit 313c according to the assigned permutation.

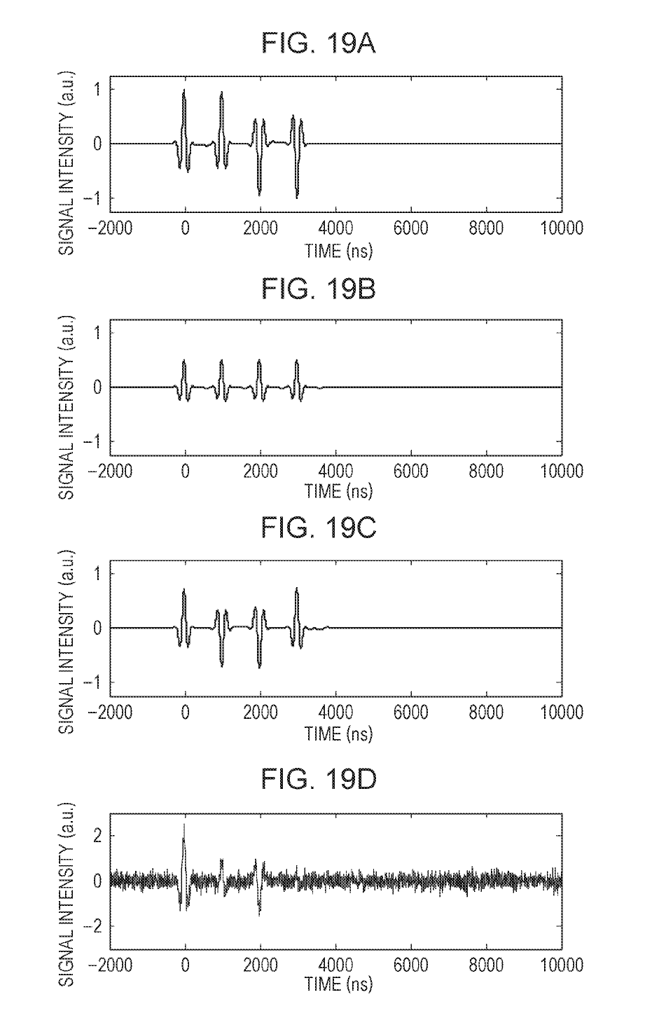

The first light source 311a is driven by a driving current generated by the first driving unit 313a according to the information on the third coding sequence {aᵢ³}. The generated light is applied to the point-like light absorber (sound source) via the first optical system 312a. A received signal obtained by receiving photoacoustic waves generated at that time with the receiving unit 120 has a waveform as illustrated in FIG. 19A.

The second light source 311b is driven by a driving current generated by the second driving unit 313b according to the information on the fourth coding sequence {aᵢ⁴}. The generated light is applied to the point-like light absorber (sound source) via the second optical system 312b. A received signal obtained by receiving photoacoustic waves generated at that time with the receiving unit 120 has a waveform as illustrated in FIG. 19B.

The third light source 311c is driven by a driving current generated by the third driving unit 313c according to the information on the first coding sequence {aᵢ¹}. The generated light is applied to the point-like light absorber (sound source) via the third optical system 312c. A received signal obtained by receiving photoacoustic waves generated at that time with the receiving unit 120 has a waveform as illustrated in FIG. 19C.

### [S6. Acquisition of Received Signal Corresponding to Third Permutation Element]

When the light outputs from the first light source 311a, the second light source 311b, and third light source 311c are synchronized (when the lights are emitted at substantially the same timing), the signal received by the receiving unit 120 is the sum of the signals illustrated in FIGS. 19A, 19B, and 19C. The received signal S3(t) obtained at that time is illustrated in FIG. 19D. In other words, the signal S3(t) is obtained by applying intensity-modulated light of the first wavelength corresponding to the third coding sequence {aᵢ³}, intensity-modulated light of the second wavelength corresponding to the fourth coding sequence {aᵢ⁴}, and intensity-modulated light of the third wavelength corresponding to the first coding sequence {aᵢ¹} at substantially the same timing. To describe the effect of reducing noise, noise of a mean value of 0 and a standard deviation of 0.2 is added.

### [S7. Application of Intensity-Modulated Light Corresponding to Fourth Permutation Element]

The control unit 153 transmits the information on the fourth coding sequence {aᵢ⁴} to the first driving unit 313a according to the assigned permutation. The control unit 153 transmits the information on the third coding sequence {aᵢ³} to the second driving unit 313b according to the assigned permutation. The control unit 153 transmits the information on the second coding sequence {aᵢ²} to the third driving unit 313c according to the assigned permutation.

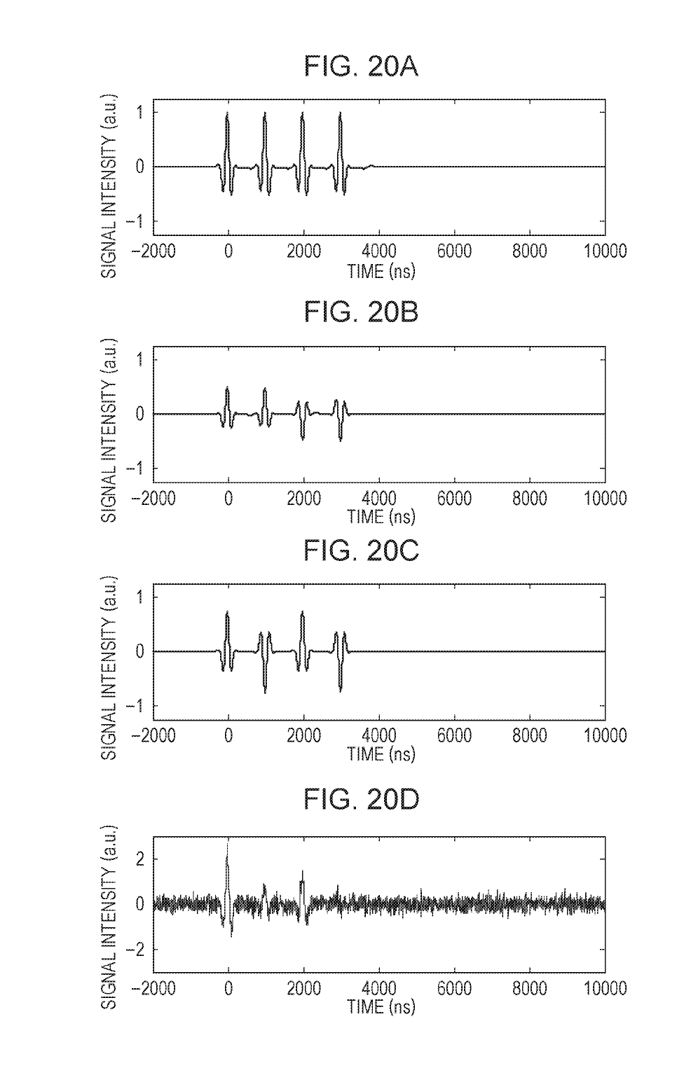

The first light source 311a is driven by a driving current generated by the first driving unit 313a according to the information on the fourth coding sequence {aᵢ⁴}. The generated light is applied to the point-like light absorber (sound source) via the first optical system 312a. A received signal obtained by receiving photoacoustic waves generated at that time with the receiving unit 120 has a waveform as illustrated in FIG. 20A.

The second light source 311b is driven by a driving current generated by the second driving unit 313b according to the information on the third coding sequence {aᵢ³}. The generated light is applied to the point-like light absorber (sound source) via the second optical system 312b. A received signal obtained by receiving photoacoustic waves generated at that time with the receiving unit 120 has a waveform as illustrated in FIG. 20B.

The third light source 311c is driven by a driving current generated by the third driving unit 313c according to the information on the second coding sequence {aᵢ²}. The generated light is applied to the point-like light absorber (sound source) via the third optical system 312c. A received signal obtained by receiving photoacoustic waves generated at that time with the receiving unit 120 has a waveform as illustrated in FIG. 20C.

### [S8. Acquisition of Received Signal Corresponding to Fourth Permutation Element]

When the light outputs from the first light source 311a, the second light source 311b, and third light source 311c are synchronized (when the lights are emitted at substantially the same timing), the signal received by the receiving unit 120 is the sum of the signals illustrated in FIGS. 20A, 20B, and 20C. The received signal S₄(t) obtained at that time is illustrated in FIG. 20D. In other words, the signal S₄(t) is obtained by applying intensity-modulated light of the first wavelength corresponding to the fourth coding sequence {aᵢ⁴}, intensity-modulated light of the second wavelength corresponding to the third coding sequence {aᵢ³}, and intensity-modulated light of the third wavelength corresponding to the second coding sequence {aᵢ²} at substantially the same timing. To describe the effect of reducing noise, noise of a mean value of 0 and a standard deviation of 0.2 is added.

In FIGS. 17A to 20D, actually, a time corresponding to the time the photoacoustic wave is propagated from the point-like light absorber to the receiving unit 120 (about 2 μs) shifts, but the time is ignored in this graph.

### [S9. Encoding]

A method for decoding a coded received signal with the operating unit 151 in the computer 150 will be described.

The operating unit 151 performs a decoding process according to Expressions 7 to 9 to obtain decoded signals DS₁(t), DS₂(t), and DS₃(t) corresponding to the individual wavelengths, where Δt is the time interval of the reference timing. The four coding sequences are used for decoding in the order determined by the same permutations as those in assignment to the lights of the first, second, and third wavelengths.

$$\begin{matrix} [ Math . 9 ] & \\ {DS}_{1} ( t ) = {\sum }_{i = 1}^{N} {S}_{1} ( t + ( i - 1 ) Δ t ) \cdot {a}_{i}^{1} + {\sum }_{i = 1}^{N} {S}_{2} ( t + ( i - 1 ) Δ t ) \cdot {a}_{i}^{2} + {\sum }_{i = 1}^{N} {S}_{3} ( t + ( i - 1 ) Δ t ) \cdot {a}_{i}^{3} + {\sum }_{i = 1}^{N} {S}_{4} ( t + ( i - 1 ) Δ t ) \cdot {a}_{i}^{4} & ( 7 ) \\ [ Math . 10 ] & \\ {DS}_{2} ( t ) = {\sum }_{i = 1}^{N} {S}_{1} ( t + ( i - 1 ) Δ t ) \cdot {a}_{i}^{2} + {\sum }_{i = 1}^{N} {S}_{2} ( t + ( i - 1 ) Δ t ) \cdot {a}_{i}^{1} + {\sum }_{i = 1}^{N} {S}_{3} ( t + ( i - 1 ) Δ t ) \cdot {a}_{i}^{4} + {\sum }_{i = 1}^{N} {S}_{4} ( t + ( i - 1 ) Δ t ) \cdot {a}_{i}^{3} & ( 8 ) \\ [ Math . 11 ] & \\ {DS}_{3} ( t ) = {\sum }_{i = 1}^{N} {S}_{1} ( t + ( i - 1 ) Δ t ) \cdot {a}_{i}^{3} + {\sum }_{i = 1}^{N} {S}_{2} ( t + ( i - 1 ) Δ t ) \cdot {a}_{i}^{4} + {\sum }_{i = 1}^{N} {S}_{3} ( t + ( i - 1 ) Δ t ) \cdot {a}_{i}^{1} + {\sum }_{i = 1}^{N} {S}_{4} ( t + ( i - 1 ) Δ t ) \cdot {a}_{i}^{2} & ( 9 )\end{matrix}$$

Generalization of Expressions 7 to 9 results in Expression 10, where N is the code length, and K is the number of coding sequences orthogonal o each other (the number of permutation elements).

$$\begin{matrix}[ Math . 12 ] & \\ {DS}_{m} ( t ) = {\sum }_{j = 1}^{K} {\sum }_{i = 1}^{N} {S}_{j} ( t + ( i - 1 ) Δ t ) \cdot {a}_{i}^{{g}_{m} ( j )} & ( 10 )\end{matrix}$$

where i is a natural number equal to or greater than 1, {gₙ(j)} is a permutation assigned to each of the plurality of wavelengths, Sⱼ is a received signal corresponding to a permutation element, j is a natural number equal to or greater than 1, K is the power of 2 that satisfies [K is equal to or greater than M], m is a natural number equal to or greater than 1 and equal to or less than M, M is the number of a plurality of wavelengths, t is time, and Δt is the time interval of the reference timing of the coding elements of the coding sequence.

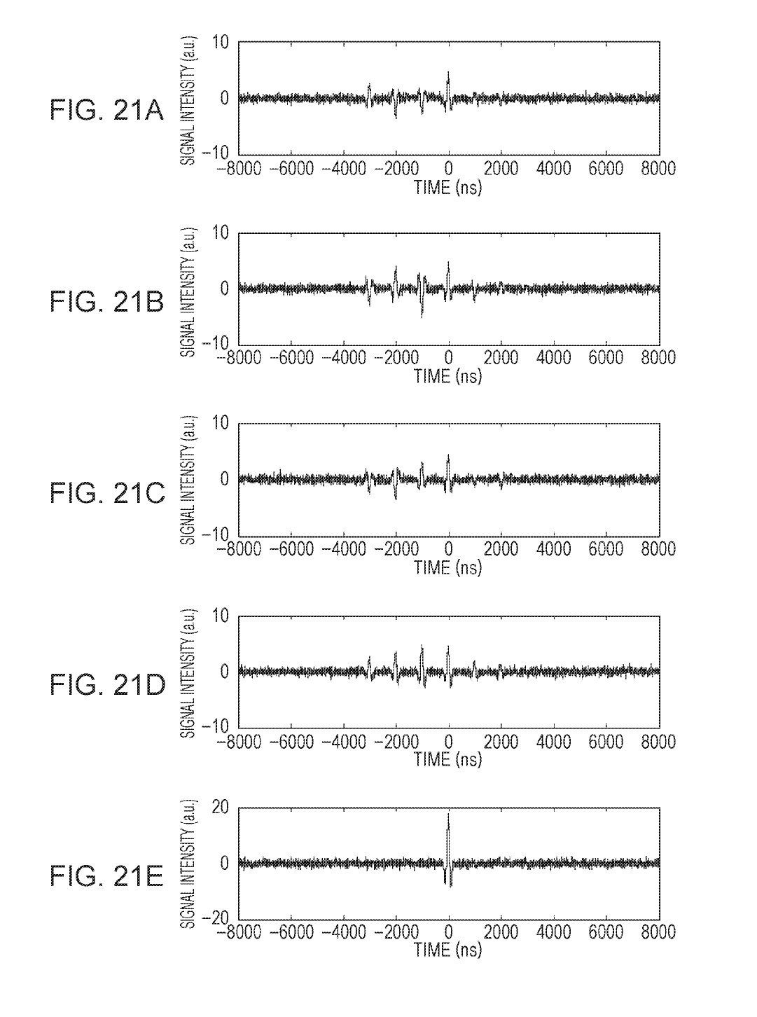

Decoding corresponding to the first wavelength will be described with reference to FIGS. 21A to 21E.

As a result of the decoding process of the first term on the right side of Expression 7 on the reception waveform (S₁) in FIG. 17D, the decoded received signal as in FIG. 21A can be obtained. As a result of the decoding process of the second term on the right side of Expression 7 on the reception waveform (S₂) in FIG. 18D, the decoded received signal as in FIG. 21B can be obtained. As a result of the decoding process of the third term on the right side of Expression 7 on the reception waveform (S₃) in FIG. 19D, the decoded received signal as in FIG. 21C can be obtained. As a result of the decoding process of the fourth term on the right side of Expression 7 on the reception waveform (S₄) in FIG. 20D, the decoded received signal as in FIG. 21D can be obtained. The sum of the signals in FIGS. 21A to 21D forms the decoded signal DS₁ corresponding to the first waveform illustrated in FIG. 21E. In other words, the peak value of the received signal DS₁ decoded according to Expression 7 is 16 times the peak value of a received signal obtained by single light irradiation, and the sidelobes are suppressed below noise.

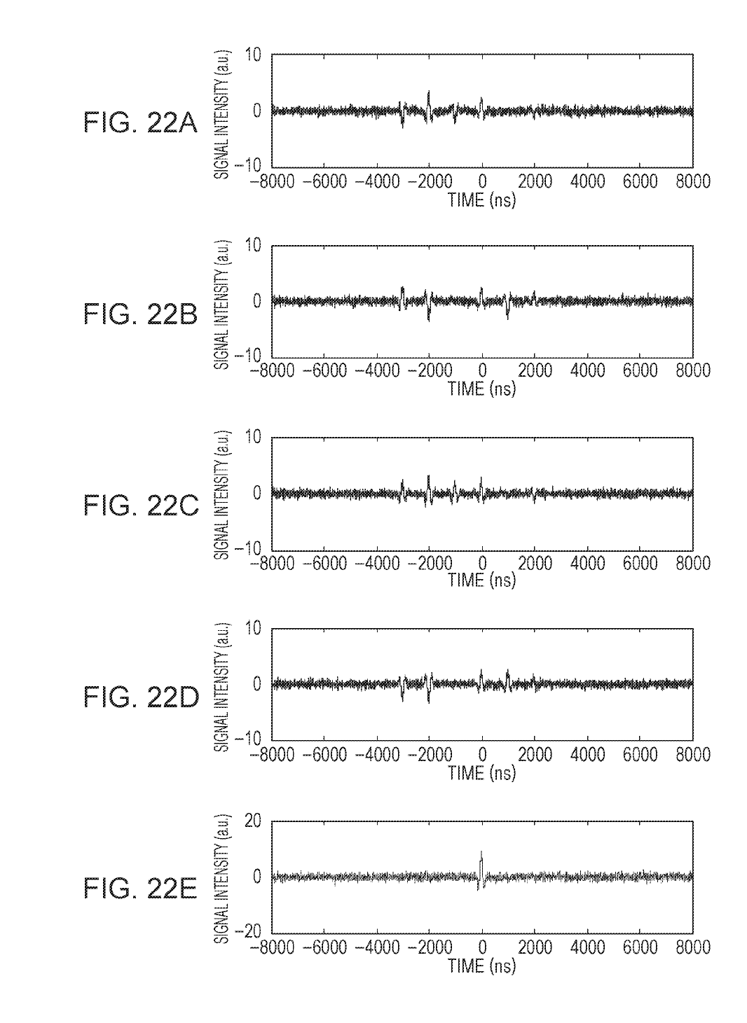

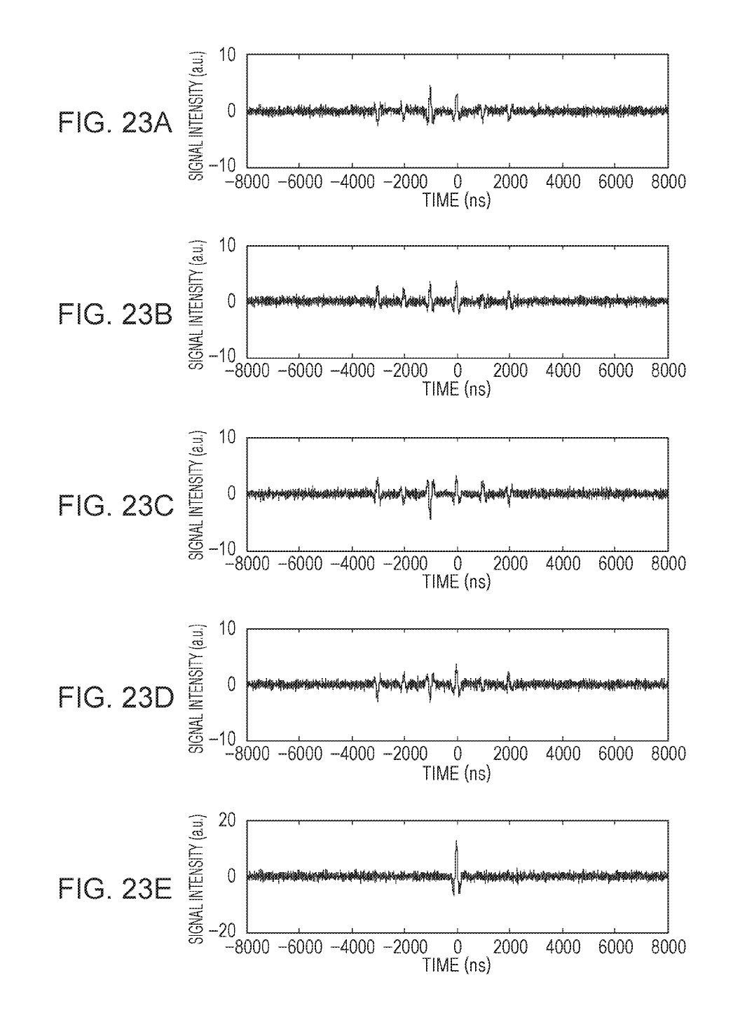

Likewise, decoding corresponding to irradiation of light of the second wavelength is illustrated in FIGS. 22A to 22E, and decoding corresponding to irradiation of light of the third wavelength is illustrated in FIGS. 23A to 23E. This shows that the peak values of the signals DS₂ and DS₃ decoded according to Expressions 8 and 9, respectively, are 16 times the peak value of a received signal obtained by single light irradiation, and the sidelobes are suppressed below noise.

A comparison of FIGS. 21E, 22E, and 23E shows that the ratio of the absorption coefficient of the absorber is stored. This indicates that information on light absorption of the object is stored even if encoding and decoding is performed. This allows spectral information, such as the hemoglobin concentration of blood, to be acquired by analyzing the decoded signals DS₁, DS₂ and DS₃ corresponding to a plurality of wavelengths.

As in the first embodiment, the control unit 153 may set the time interval of the reference timing according to the observation region (the region of interest) or the sound velocity of the object so that a signal with a high S/N ratio can be obtained in a short time. A method of reconstruction may be the same method as that of the first embodiment. The present embodiment may also employ the configuration of the driving unit 113 described in the first embodiment.

Although the present embodiment illustrates an example of acquiring decoded signals corresponding to individual three or more wavelengths, it is only required that a decoded signal corresponding to at least one of three or more wavelengths is acquired. In other words, in the present embodiment, a decoded signal corresponding to at least one of a plurality of wavelengths is acquired. This also allows a decoded signal corresponding to a desired wavelength to be acquired.

### Third Embodiment

### [Encoding and Decoding Using Small Number of Coding Sequences]

The method described in the second embodiment can improve the S/N ratio using a coding sequence with a code length of 8 instead of 4. However, this increases the number of coding sequences orthogonal to each other to 8. This requires repeating light irradiation and reception of photoacoustic waves for encoding eight times, increasing the measurement time.

In the present embodiment, an example of improving the S/N ratio while preventing an increase in measurement time by decreasing the number of coding sequences.

Consider four coding sequences {aᵢ^{k}}(k=1 to 4, i=1 to 8) orthogonal to one another and having a code length of 8:

a first coding sequence {aᵢ¹}={1, 1, −1, −1, −1, −1, 1, 1}
a second coding sequence {aᵢ²}={1, 1, −1, −1, 1, 1, −1, −1}
a third coding sequence {aᵢ³}={1, 1, 1, 1, −1, −1, −1, −1}
a fourth coding sequence {aᵢ⁴}={1, 1, 1, 1, 1, 1, 1, 1}
These are the results of assigning {1, 1} to {1} and {−1, −1} to {−1} in the coding sequences described in the second embodiment. In other words, by repeating each coding element of the four coding sequences orthogonal to each other twice, the number of coding elements is doubled. The repeat count of each coding element may be any natural number that is equal to or greater than 2.

Furthermore, consider the following four different permutations for arranging the four coding sequences in order without overlapping:

a first permutation {g₁}={{aᵢ¹}, {aᵢ²}, {aᵢ³}, {aᵢ⁴}}
a second permutation {g₂}={{aᵢ²}, {aᵢ¹}, {aᵢ⁴}, {aᵢ³}}
a third permutation {g₃}={{aᵢ³}, {aᵢ⁴}, {aᵢ²}}
a fourth permutation {g₄}={{aᵢ⁴}, {aᵢ³}, {aᵢ²}, {aᵢ¹}}
Here consider the total sum of the respective cross-correlation functions of the four coding sequences arranged according to the permutations gₚ and g_{q}, as expressed by Expression 6.

If p=q, Expression 6 takes the total sum of the autocorrelation functions. The total sum is 16, 32, and 16 in the vicinity of the peak, and O at all points other than the peak. That is,

(a¹* a¹)={1, −2, 1, −4, −3, −2, 3, 8, 3, −2, −3, −4, −1, 2, 1}
(a²* a²)={−1, −2, 1, 4, −1, −6, 1, 8, 1, −6, −1, 4, 1, −2, −1}
(a³* a³)={−1, −2, −3, −4, −1, 2, 5, 8, 5, 2, −1, −4, −3, −2, −1}
(a⁴* a⁴)={1, 2, 3, 4, 5, 6, 7, 8, 7, 6, 5, 4, 3, 2, 1} and, at any p,

$${\sum }_{j = 1}^{4} ( {a}^{{g}_{p} ( j )} * {a}^{{g}_{p} ( j )} ) = ( {a}^{1} * {a}^{1} ) + ( {a}^{2} * {a}^{2} ) + ( {a}^{3} * {a}^{3} ) + ( {a}^{4} * {a}^{4} ) = \{ 0 , 0 , 0 , 0 , 0 , 0 , 16 , 32 , 16 , 0 , 0 , 0 , 0 , 0 , 0 \} \qquad [ Math . 13 ]$$

holds.

If p≠q, Expression 6 takes the total sum of the autocorrelation functions. The total sum is 0 in all combinations of p≠q. For example, if p=1 and q=2,

(a¹* a²)={−1, −2, 1, 4, 1, −2, −1, 0, 1, 2, −1, −4, −1, 2, 1}
(a²* a¹)={1, 2, −1, −4, −1, 2, 1, 0, −1, −2, 1, 4, 1, −2, −1}
(a³* a⁴)={1, 2, 3, 4, 3, 2, 1, 0, −1, −2, −3, −4, −3, −2, −1}
(a⁴* a³)={−1, −2, −3, −4, −3, −2, −1, 0, 1, 2, 3, 4, 3, 2, 1}

$${\sum }_{j = 1}^{4} ( {a}^{{g}_{1} ( j )} * {a}^{{g}_{2} ( j )} ) = ( {a}^{1} * {a}^{2} ) + ( {a}^{2} * {a}^{1} ) + ( {a}^{3} * {a}^{4} ) + ( {a}^{4} * {a}^{3} ) = \{ 0 , 0 , 0 , 0 , 0 , 0 , 0 , 0 , 0 , 0 , 0 , 0 , 0 , 0 , 0 \} \qquad [ Math . 14 ]$$

holds.

Using such coding sequences and permutations indicating their orders in a photoacoustic imaging apparatus that uses lights of a plurality of different wavelengths allows a signal obtained from light of a certain wavelength and a signal obtained from light of the other wavelengths to be separately obtained even if the light irradiation times overlap. Thus, spectral information, such as the hemoglobin concentration of blood, can be acquired by analyzing the decoded signals corresponding to a plurality of wavelengths thus separated.

Compared with the second embodiment, if Δt<T_{tof} holds, the peak intensity of a decoded signal corresponding to each wavelength can be increased with little increase in measurement time. At that time, sidelobes also increases, but its pattern is known. Therefore, the decoded received signals are corrected as appropriate as the need arises. For example, of decoded received signals, only signals of a certain threshold or greater may be validated. Furthermore, it is known that the decoded received signals have a signal ratio of 1:2:1 at an interval of Δt. Therefore, a deconvolution filter that corrects the signals to signals of a signal ratio of 0:1:0 may be superposed thereon.

Although the present embodiment is an example of acquiring decoded signals corresponding to a plurality of wavelengths, a decoded signal corresponding to at least one of a plurality of wavelengths may be acquired. Also in this case, a decoded signal corresponding to a desired wavelength can be acquired.

### Other Embodiments

Embodiment (s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-228059, filed Nov. 24, 2016, which is hereby incorporated by reference herein in its entirety.

1. A photoacoustic imaging apparatus comprising:
a light irradiation unit;
a receiving unit; and
a processing unit,
wherein the light irradiation unit irradiates an object with first intensity-modulated light of a first wavelength corresponding to a first coding sequence and second intensity-modulated light of a second wavelength corresponding to a second coding sequence different from the first coding sequence,
wherein the receiving unit outputs a first signal by receiving photoacoustic waves generated by irradiating the object with the first and second intensity-modulated light, and
wherein the processing unit obtains at least one of a first decoded signal corresponding to the first wavelength and a second decoded signal corresponding to the second wavelength by performing a decoding process on the first signal based on information on the first and second coding sequences.

2. The photoacoustic imaging apparatus according to claim 1, wherein an irradiation period of the first intensity-modulated light and an irradiation period of the second intensity-modulated light at least partly overlap with each other.

3. The photoacoustic imaging apparatus according to claim 1, wherein the processing unit acquires the first and second decoded signals and acquires spectral information based on the first and second decoded signals.

4. The photoacoustic imaging apparatus according to claim 3, wherein the spectral information comprises oxygen saturation.

5. The photoacoustic imaging apparatus according to claim 1, wherein each of the first and second coding sequences includes a positive coding element and a negative coding element.

6. The photoacoustic imaging apparatus according to claim 5, wherein the light irradiation unit comprises:
a light source comprising a semiconductor laser or a light-emitting diode; and
a driving unit configured to input a driving current to the light source,
wherein the driving unit inputs a first driving current for generating positive intensity-modulated light corresponding to the positive coding element to the light source in accordance with reference timing corresponding to the positive coding element, and
wherein the driving unit inputs a second driving current for generating negative intensity-modulated light corresponding to the negative coding element to the light source in accordance with the reference timing corresponding to the positive coding element.

7. The photoacoustic imaging apparatus according to claim 6, wherein the driving unit comprises:
a first driving unit configured to input the first driving current to the light source; and
a second driving unit configured to input the second driving current to the light source.

8. The photoacoustic imaging apparatus according to claim 1, wherein the light irradiation unit comprises:
a first light source comprising a semiconductor laser or a light-emitting diode that emits light of the first wavelength; and
a second light source comprising a semiconductor laser or a light-emitting diode that emits light of the second wavelength.

9. The photoacoustic imaging apparatus according to claim 1,
wherein the light irradiation unit irradiates the object with third intensity-modulated light of the first wavelength corresponding to a third coding sequence and fourth intensity-modulated light of the second wavelength corresponding to a fourth coding sequence different from the third coding sequence,
wherein the receiving unit outputs a second signal by receiving photoacoustic waves generated by irradiating the object with the third and fourth intensity-modulated light,
wherein the processing unit obtains the first decoded signal corresponding to the first wavelength by performing a decoding process on the first and second signals based on information on the first and third coding sequences, and
wherein the processing unit obtains the second decoded signal corresponding to the second wavelength by performing a decoding process on the first and second signals based on information on the second and fourth coding sequences

10. The photoacoustic imaging apparatus according to claim 9,
wherein the first and third coding sequences are first complementary codes having complementary relationship,
wherein the second and fourth coding sequences are second complementary codes having complementary relationship, and
wherein a sum of a cross-correlation function of the first coding sequence and the second coding sequence and a cross-correlation function of the third coding sequence and the fourth coding sequence is zero.

11. The photoacoustic imaging apparatus according to claim 9,
wherein an irradiation period of the first intensity-modulated light and an irradiation period of the second intensity-modulated light at least partly overlap with each other,
wherein an irradiation period of the third intensity-modulated light and an irradiation period of the fourth intensity-modulated light at least partly overlap with each other, and
wherein the irradiation periods of the first and second intensity-modulated light and the irradiation periods of the third and fourth intensity-modulated light do not overlap with each other.

12. The photoacoustic imaging apparatus according to claim 9, wherein the processing unit acquires the first decoded signal DS₁(t) and the second decoded signal DS₂(t) by performing the decoding process according to a following expression:
$${DS}_{1} ( t ) = {\sum }_{i = 1}^{N} {S}_{1} ( t + ( i - 1 ) Δ t ) \cdot {a}_{i} + {\sum }_{i = 1}^{N} {S}_{2} ( t + ( i - 1 ) Δ t ) \cdot {c}_{i} \\ {DS}_{2} ( t ) = {\sum }_{i = 1}^{N} {S}_{1} ( t + ( i - 1 ) Δ t ) \cdot {b}_{i} + {\sum }_{i = 1}^{N} {S}_{2} ( t + ( i - 1 ) Δ t ) \cdot {d}_{i} \qquad [ Math . 1 ]$$
where Δt is a time interval of a reference timing corresponding to a coding element, {aᵢ}, {bᵢ}, {cᵢ}, and {dᵢ} (i is an integer from 1 to N, where N is a code length, and each coding element is 1 or −1) are respectively the first, second, third, and fourth coding sequences, S₁(t) is the first signal, and S₂(t) is the second signal.

13. An photoacoustic imaging apparatus comprising:
a light irradiation unit;
a receiving unit; and
a processing unit,
wherein the light irradiation unit irradiates an object with intensity-modulated lights of a plurality of wavelengths corresponding to a plurality of coding sequences,
wherein the receiving unit outputs signals by receiving photoacoustic waves generated by irradiation with the intensity-modulated lights of the plurality of wavelengths, and
wherein the processing unit obtains a decoded signal corresponding to at least one of the plurality of wavelengths by performing a decoding process on each signal based on information on the plurality of coding sequences.

14. The photoacoustic imaging apparatus according to claim 13, wherein the processing unit acquires the decoded signal DSₘ(t) (m is a natural number equal to or greater than 1 and equal to or less than M, where M is the number of the plurality of wavelengths, and t is time) by performing the decoding process on each signal according to a following expression:
$${DS}_{m} ( t ) = {\sum }_{j = 1}^{K} {\sum }_{i = 1}^{N} {S}_{j} ( t + ( i - 1 ) Δ t ) \cdot {a}_{i}^{{g}_{m} ( j )} \qquad [ Math . 2 ]$$
where ({aᵢ^{k}} is a coding sequence, i is a natural number equal to or greater than 1, N is a code length, Δt is a time interval of a reference timing of a coding element of a coding sequence, {gₘ(j)} is a permutation that determines an order of a coding sequence assigned to each of the plurality of wavelengths, j is a natural number equal to or greater than 1, K is the number of elements of the permutation and the power of 2 that satisfies [K is equal to or greater than M], and Sⱼ is the signal corresponding to a permutation element.

15. The photoacoustic imaging apparatus according to claim 13, wherein the plurality of coding sequences are orthogonal to each other.

16. The photoacoustic imaging apparatus according to claim 13, wherein the plurality of wavelengths comprises three or more wavelengths.

17. A method for acquiring information, comprising:
irradiating an object with first intensity-modulated light of a first wavelength corresponding to a first coding sequence and second intensity-modulated light of a second wavelength corresponding to a second coding sequence different from the first coding sequence;
outputting a first signal by receiving photoacoustic waves generated by irradiating the object with the first and second intensity-modulated light; and
obtaining at least one of a first decoded signal corresponding to the first wavelength and a second decoded signal corresponding to the second wavelength by performing a decoding process on the first signal based on information on the first and second coding sequences.

18. A method for acquiring information, comprising:
irradiating an object with intensity-modulated lights of a plurality of wavelengths corresponding to a plurality of coding sequences;
outputting signals by receiving photoacoustic waves generated by irradiation with the intensity-modulated lights of the plurality of wavelengths; and
obtaining a decoded signal corresponding to at least one of the plurality of wavelengths by performing a decoding process on each signal based on information on the plurality of coding sequences.

19. A non-transitory computer-readable medium including a program for causing a computer to execute the method for acquiring information according to claim 17.
